(19) United States
(12) Patent Application Publication
(10) Pub. No.: US 2024/0049725 A1
(43) Pub. Date: Feb. 15, 2024

(54) COMPOSITIONS COMPRISING CYCLOTIDES AND OTHER INSECTICIDAL PEPTIDES AND USES THEREOF

(71) Applicant: SYNGENTA CROP PROTECTION AG, Basel (CH)

(72) Inventors: Aurelien BIGOT, Stein (CH); Fides BENFATTI, Stein (CH); David J. CRAIK, St Lucia (AU); Yen-Hua HUANG, St Lucia (AU); Quentin KAAS, St Lucia (AU); Mark SCHIEBLER, Stein (CH); Conan WANG, St Lucia (AU)

(73) Assignee: SYNGENTA CROP PROTECTION AG, Basel (CH)

(21) Appl. No.: 18/258,636

(22) PCT Filed: Dec. 21, 2021
(86) PCT No.: PCT/EP2021/087104

§ 371 Date: Jun. 21, 2023

(30) Foreign Application Priority Data
Dec. 21, 2020 (EP) 20216143.6

(51) Int. Cl. A01N 63/50; A01P 7/04

(52) U.S. Cl. CPC A01N 63/50; A01P 7/04

(57) ABSTRACT

The present invention relates to an insecticidal composition comprising: a. at least one cyclic peptide comprising an amino acid sequence of any of: SEQ ID NOs: 1, 2 or 6, or an amino acid sequence that is at least 90%, 91%, 92%, 93%, 94%, 95%, 96%, 97%, 98% or 99% identical to any of SEQ ID NOs: 1, 2 or 6; and b. at least one second peptide comprising an amino acid sequence of any of SEQ ID NOs: 3 to 5, or an amino acid sequence that is at least 90%, 91%, 92%, 93%, 94%, 95%, 96%, 97%, 98% or 99% identical to any of SEQ ID NOs: 3 to 5, or a variant of a peptide comprising an amino acid sequence of SEQ ID NO: 3, wherein the amino acid sequence has an amino acid variant on position N29, and which retains the biological activity of the peptide.

### FIELD OF THE INVENTION

The present invention describes novel insecticidal peptide compositions, their application to plants, methods for the delivery of the compositions, methods of controlling insect infestations and occurrence by use of the compositions, and uses of the compositions for controlling insect populations.

### BACKGROUND OF THE INVENTION

Crop loss caused by arthropods and in particular by insects has a major global economic impact. Reduction in yields of wheat, rice, maize, potato and soybean caused by damage associated with 137 pathogens and pests amounts to 17.2%-30% on average (Savary et al., Nature Ecology & Evolution, 2019). This causes a severe strain on the ability to meet the increasing global demand for food production.

Chemical insecticides were introduced during the second half of the 20^{th} century and are to this date still the most important method for reducing damage caused by insect pests. However, a disadvantage of agrochemicals is considered to be their impact on the environment.

Biopesticides are currently being investigated as a safe and effective alternatives to chemical pesticides. These include naturally occurring substances that control pests, such as biochemical pesticides. Insecticidal toxins derived from natural enemies are of particular interest. These include (neuro)toxins produced by spiders, scorpions and sea anemones, (endo)toxins produced by microorganisms such as Bacillus thuringiensis, Xenorhabdus nematophilia and Photorhabdus luminescens, and plant-produced toxins such as defensins.

An issue with these insecticidal toxins is that, when directly applied, there remains a difficulty to reach their target site of interest. The most practical route of application of many of these toxins is through ingestion of the toxin by a target insect. However, the insect's digestive enzymes and the physical barriers of the alimentary canal can prevent uptake of a sufficient amount of insecticidal toxin to reach the target site of interest, and thereby greatly reduces the efficacy of the toxins.

Currently, the most widely applied and most successful bioinsecticides are 5-endotoxins produced by B. thuringiensis. After ingestion by an insect, these toxins bind to the gut endothelium, form cation-selective channels, and cause cell lysis, which then leads to death of the insect. However, a growing concern is the increasing resistance that insect species are developing to these toxins.

Compositions and variants of insecticidal peptide toxins are for example described in WO2013134734A2 and WO2020056315A1.

WO2013134734A2 discloses a composition comprising two types of insecticidal protein or peptides wherein one type is a Pore Forming Insecticidal Protein and the other type is a Cysteine Rich Insecticidal Peptide. However, The Pore Forming Insecticidal Proteins described in WO2013134734A2 comprise only proteins derived from B. thuringiensis. The disclosed compositions always include at least B. thuringiensis toxin peptide variants.

WO2020056315A1 discloses Av3 insecticidal toxin peptide variants that can be combined with other insecticidal toxin peptides such as AaIT1 and/or B. thuringiensis toxin peptides.

Accordingly, the above compositions disclosures rely on insecticidal toxins originating from B. thuringiensis, which are likely to be subject to rising resistance issues against these toxins.

Hence, there remains a need for insecticidal peptide compositions, preferably those that offer additional options enabling improved efficacy. There further remains a need for insecticidal peptide compositions that may be delivered to insects via ingestion.

### SUMMARY OF THE INVENTION

The compositions, methods and uses of the present invention enhance the efficacy of insecticidal peptide toxins. As a result they are more useful when combined as such than when applied alone and provide an important improvement in the practical application of biopesticides.

Accordingly, a first aspect of the present invention relates to an insecticidal composition comprising:
a. at least one cyclic peptide comprising an amino acid sequence of: SEQ ID NOs: 1, 2 or 6, or an amino acid sequence that is at least 90%, 91%, 92%, 93%, 94%, 95%, 96%, 97%, 98% or 99% identical to any of SEQ ID NOs: 1, 2 or 6; and
b. at least one second peptide comprising an amino acid sequence of any of SEQ ID NOs: 3 to 5, or an amino acid sequence that is at least 90%, 91%, 92%, 93%, 94%, 95%, 96%, 97%, 98% or 99% identical to any of SEQ ID NOs: 3 to 5, or a variant of a peptide comprising an amino acid sequence of SEQ ID NO: 3, wherein the amino acid sequence has an amino acid variant on position N29, and which retains the biological activity of the peptide.

In a further aspect, the present invention provides for a composition according to the invention wherein the cyclic peptide and/or the second peptide exhibit insecticidal activity, when in contact with an insect, in particular when ingested by an insect.

In again a further aspect, the invention provides for a plant, plant tissue or plant propagation material comprising a composition according to the invention or having a composition according to the invention applied thereto.

In another aspect, the invention provides a composition comprising an insecticidally effective amount of the peptides according to the invention and a suitable carrier or diluent therefor.

In yet a further aspect, the invention provides a method for delivering a composition to an insect, the method comprising applying the composition according to the invention to a plant, plant locus, plant seed or otherwise plant propagation material, to obtain a treated material.

In yet a further aspect, the invention provides a method for delivering a composition to an insect, the method comprising applying the composition according to the invention.

In yet a further aspect, the invention provides for a method for controlling insects, comprising applying a composition according to the invention to an insect or its environment, a plant, plant tissue or plant propagation material or the locus where the plant or plant propagation material is planted.

In yet a further aspect, the invention provides for a use of a composition for controlling insect infestations and/or limiting plant damage.

### BRIEF DESCRIPTION OF THE SEQUENCE LISTING

This specification includes a sequence listing of 7 sequences.
SEQ ID NOs: 1 and 2 are sequences derived from or having a plant origin, namely Oldenlandia affinis.
SEQ ID NOs: 3 is a sequence derived from or having a spider origin, namely Hadronyche versuta.
SEQ ID NO: 4 is a sequence derived from or having an anemone origin, namely Anemonia sulcata.
SEQ ID NO: 5 is a sequence derived from or having a spider origin, namely Hadronyche versuta to which the amino acids GS are added.
SEQ ID NO: 6 is a mutated sequence of SEQ ID NO: 1.
SEQ ID NO: 33 is a sequence derived from or having a spider origin, namely Atrax robustus.

### DETAILED DESCRIPTION OF THE INVENTION

The present invention describes new insecticidal peptide compositions, their application to plants, their expression in plants, methods for delivery of the compositions, methods for controlling insect infestations by the compositions, and the use of the compositions for controlling insect infestations. The insecticidal peptide compositions have an increased insecticidal effect when applied as a composition compared to when applied separately.

The present invention accordingly relates to an insecticidal composition comprising:
a. at least one cyclic peptide comprising an amino acid sequence of any of SEQ ID NOs: 1, 2 or 6, or homologues thereof, or an amino acid sequence that is at least 65%, 70%, 75%, 80%, 85%, 90%, 91%, 92%, 93%, 94%, 95%, 96%, 97%, 98% or 99% identical to any of SEQ ID NOs: 1, 2 or 6; and b. at least one second peptide comprising an amino acid sequence of any of SEQ ID NOs: 3 to 5, or homologues thereof, or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5, or a variant of a peptide comprising an amino acid sequence of SEQ ID NO: 3, wherein the amino acid sequence has an amino acid variant on position N29, and which substantially retains the biological activity of the peptide.

In this application we describe that compositions comprising these specific combinations of peptides have an increased insecticidal effect, thereby improving the efficacy of each insecticidal peptide and therefore requiring less of each peptide when used to control insects when compared to when each peptide is used individually.

The at least one cyclic peptide are the first component of the composition and are advantageously selected from the family of cyclotides.

Insecticidal is herein understood to mean to kill, to destroy, to retard or prevent growth, movement or feeding or to inhibit reproduction of insects by a compound or peptide.

Kalata B1 (kB1, SEQ ID NO: 1) belongs to the Möbius family of cyclotides. Cyclotides are stable plant-derived cyclic peptides stabilized by three disulfide bridges that form a cysteine knot and they typically contain 28 to 37 peptides. Möbius cyclotides are characterized by having a cis-proline in loop 5 that induces a local 1800 backbone twist presenting a characterizing structure. The primary function of cyclotides in plants is that of host defence molecules.

Kalata B1 (kB1, SEQ ID NO: 1) belongs to the Möbius family of cyclotides. Kalata B1 and its related family member kalata B2 (kB2, SEQ ID NO: 2) both have potent insecticidal activity against the larvae of Helicoverpa armigera (Jennings et al, 2005). In addition, cyclotides in general have been shown to not only have insecticidal activity, but also toxic activity against nematodes, trematodes and mollusks such as kalataB1[V25K] (kB1[V25K], SEQ ID NO: 6) (Huang et al, 2010; Weidmann and Craik, 2016).

Kalata B1 and B2 are thought to disrupt cell membranes by binding to phosphatidylethanolamine and to form lipidic toroidal pores (Cranfield et al., 2017). This is in contrast to B. thuringiensis δ-endotoxins which, after cleavage in the midgut and oligomerization, form transmembrane ionic pores via binding to a membrane receptor such as aminopeptidase N or alkaline phosphatase. Since Möbius cyclotides such as kalata B1 and B2 bind to an essential structural element of the cell membrane, which is not directly genetically encoded, it will be much more difficult for a targeted organism to develop resistance against the cell disrupting effect of kalata B1 and B2 than B. thuringiensis δ-endotoxins that require interactions with multiple proteins expressed by the targeted organism.

Peptide toxins derived from natural sources such as SEQ ID NOs 3 to 5 (respectively Hv1a, Av3, Spear®-T (GS-ω/κ-Hxtx-Hv1 h) have been described in scientific literature to show pesticidal activity against various pests. The efficacy of these peptides can be increased when combined with Möbius cyclotides.

Hv1a is a 37-residue insecticidal spider-venom ω-hexatoxin peptide. Hv1a is a positive allosteric modulator of the insect nicotinic acetylcholine receptor. It is highly toxic to a variety of insect pests such as those from the orders Lepidoptera, Coleoptera, Diptera and Dictyoptera but nontoxic to mammals

Av3 is a small insecticidal sea anemone neurotoxin peptide. Av3 selectively inhibits inactivation of arthropod and crustacean, but not mammalian, voltage-gated sodium channels and is shown to be toxic to crustaceans, cockroaches, blow fly larvae and Helicoverpa armigera.

Spear®-T is a biological insecticide developed and marketed by Vestaron. It contains the peptide toxin GS-ω/κ-Hxtx-Hv1 h as its active ingredient. GS-ω/κ-Hxtx-Hv1 h is classified by the Insect Resistance Action Committee in the group 32 as an allosteric modulator of the insect nicotinic receptor at a novel site unrelated to IRAC group 4 and group 5. It is marketed as being able to control aphids, spider mites, broad mites, thrips and whiteflies.

In addition to providing increased access to the endothelial cells, cyclotides may also provide increased access to underlying structures of the insect such as the hemolymph and the nervous system. In turn, access to target sites of interest will be facilitated for insecticidal (neuro)toxins such as SEQ ID NOs: 3 to 5, thereby enhancing their insecticidal effects.

Various cyclotides have a high amino acid sequence similarity compared to kB1 and kB2. Homology among peptides is typically inferred from their amino acid sequence similarity. Significant similarity between two sequences points towards the fact that those sequences can be related through evolutionary changes from a common ancestral sequence, thereby being homologues. Highly homologous peptides or peptides having a high amino acid sequence similarity as such are usually considered to have similar functions and/or effects. Such peptides are therefore considered to be insecticidal by themselves and to also enhance the efficacy of other insecticidal peptides such as those comprising SEQ ID NOs 3 to 5.

In co-pending application European patent application no. EP20216063.6, applicant has found that variants of peptides comprising an amino acid sequence of SEQ ID NO: 3, wherein the amino acid sequence has an amino acid variant on position N29, substantially retain the biological activity of the peptide, while also having an increased stability at alkaline pH compared to the non-variant peptide.

Preferably, the at least one second peptide, preferably with insecticidal activity, comprises an amino acid sequence of any of: SPTCIPSGQPCPYNENCCSQSCTFKENENGNTVKRCD (SEQ ID NO: 3 (Hv1a)), RSCCPCYWGGCPWGQNCYPEGCSGPKV (SEQ ID NO: 4 (Av3)), GSQYCVPVDQPCSLNTQPCCDDATCTQERNENGHTVYYCRA (SEQ ID NO: 5 (GS-ω/κ-Hxtx-Hv1 h)), or homologues thereof, or an amino acid sequence that is at least 65%, 70%, 75%, 80%, 85%, 90%, 91%, 92%, 93%, 94%, 95%, 96%, 97%, 98% or 99% identical to any of SEQ ID NO: 3, SEQ ID NO: 4 or SEQ ID NO: 5.

A further embodiment of the present invention relates to a composition comprising:
a. at least one cyclic peptide comprising an amino acid sequence of any of:
GLPVCGETCVGGTCNTPGCTCSWPVCTRN (SEQ ID NO: 1),
GLPVCGETCFGGTCNTPGCSCTWPICTRD (SEQ ID NO: 2) or
GLPVCGETCVGGTCNTPGCTCSWPKCTRN (SEQ ID NO: 6); and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5).

A further embodiment of the present invention relates to a composition comprising:
a. at least one cyclic peptide comprising an amino acid sequence of any of:
GLPVCGETCVGGTCNTPGCTCSWPVCTRN (SEQ ID NO: 1),
GLPVCGETCFGGTCNTPGCSCTWPICTRD (SEQ ID NO: 2) or
GLPVCGETCVGGTCNTPGCTCSWPKCTRN (SEQ ID NO: 6); and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5) or a variant of a peptide comprising an amino acid sequence of SEQ ID NO: 3, wherein the amino acid sequence has an amino acid variant on position N29, and which substantially retains the biological activity of the peptide.

To clarify, “a variant of a peptide comprising an amino acid sequence of SEQ ID NO: 3, wherein the amino acid sequence has an amino acid variant on position N29, and which substantially retains the biological activity of the peptide” is understood to mean herein: “a variant of a peptide comprising an amino acid sequence of SEQ ID NO: 3, wherein the amino acid sequence has an amino acid variant on position N29, and wherein the amino acid sequence having an amino acid variant on position N29 substantially retains the biological activity of the peptide”.

Similarly, “a variant of a peptide comprising an amino acid sequence of SEQ ID NO: 3, wherein the amino acid sequence has an amino acid variant on position N29, and which retains the biological activity of the peptide” is understood to mean herein: “a variant of a peptide comprising an amino acid sequence of SEQ ID NO: 3, wherein the amino acid sequence has an amino acid variant on position N29, and wherein the amino acid sequence having an amino acid variant on position N29 retains the biological activity of the peptide”.

Yet a further embodiment of the present invention relates to a composition comprising:
a. at least one cyclic peptide comprising an amino acid sequence of any of:
GLPVCGETCVGGTCNTPGCTCSWPVCTRN (SEQ ID NO: 1),
GLPVCGETCFGGTCNTPGCSCTWPICTRD (SEQ ID NO: 2) or
GLPVCGETCVGGTCNTPGCTCSWPKCTRN (SEQ ID NO: 6), or homologues thereof; and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5), or homologues thereof.

Yet a further embodiment of the present invention relates to a composition comprising:
a. at least one cyclic peptide comprising an amino acid sequence of any of:
GLPVCGETCVGGTCNTPGCTCSWPVCTRN (SEQ ID NO: 1),
GLPVCGETCFGGTCNTPGCSCTWPICTRD (SEQ ID NO: 2) or
GLPVCGETCVGGTCNTPGCTCSWPKCTRN (SEQ ID NO: 6), or homologues thereof; and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5), or homologues thereof, or a variant of a peptide comprising an amino acid sequence of SEQ ID NO: 3, wherein the amino acid sequence has an amino acid variant on position N29, and which substantially retains the biological activity of the peptide.

Another embodiment of the present invention relates to a composition comprising:
a. at least one cyclic peptide comprising an amino acid sequence of any of:
GLPVCGETCVGGTCNTPGCTCSWPVCTRN (SEQ ID NO: 1),
GLPVCGETCFGGTCNTPGCSCTWPICTRD (SEQ ID NO: 2) or
GLPVCGETCVGGTCNTPGCTCSWPKCTRN (SEQ ID NO: 6), or an amino acid sequence that is at least 65%, 70%, 75%, 80%, 85%, 90%, 91%, 92%, 93%, 94%, 95%, 96%, 97%, 98% or 99% identical to any of SEQ ID NO: 1, 2 or 6; and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5), or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5.

Another embodiment of the present invention relates to a composition comprising:
a. at least one cyclic peptide comprising an amino acid sequence of any of:
GLPVCGETCVGGTCNTPGCTCSWPVCTRN (SEQ ID NO: 1),
GLPVCGETCFGGTCNTPGCSCTWPICTRD (SEQ ID NO: 2) or
GLPVCGETCVGGTCNTPGCTCSWPKCTRN (SEQ ID NO: 6), or an amino acid sequence that is at least 65%, 70%, 75%, 80%, 85%, 90%, 91%, 92%, 93%, 94%, 95%, 96%, 97%, 98% or 99% identical to any of SEQ ID NO: 1, 2 or 6; and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5), or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. at least one cyclic peptide comprising an amino acid sequence of:
GLPVCGETCVGGTCNTPGCTCSWPVCTRN (SEQ ID NO: 1),
GLPVCGETCFGGTCNTPGCSCTWPICTRD (SEQ ID NO: 2) or
GLPVCGETCVGGTCNTPGCTCSWPKCTRN (SEQ ID NO: 6), or homologues thereof, or an amino acid sequence that is at least 65%, 70%, 75%, 80%, 85%, 90%, 91%, 92%, 93%, 94%, 95%, 96%, 97%, 98% or 99% identical to any of SEQ ID NO: 1, 2 or 6; and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5), or homologues thereof, or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5.

An embodiment of the present invention relates to a composition comprising:
a. at least one cyclic peptide comprising an amino acid sequence of:
GLPVCGETCVGGTCNTPGCTCSWPVCTRN (SEQ ID NO: 1),
GLPVCGETCFGGTCNTPGCSCTWPICTRD (SEQ ID NO: 2) or
GLPVCGETCVGGTCNTPGCTCSWPKCTRN (SEQ ID NO: 6), or homologues thereof, or an amino acid sequence that is at least 65%, 70%, 75%, 80%, 85%, 90%, 91%, 92%, 93%, 94%, 95%, 96%, 97%, 98% or 99% identical to any of SEQ ID NO: 1, 2 or 6; and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5), or homologues thereof, or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5, or a variant of a peptide comprising an amino acid sequence of SEQ ID NO: 3, wherein the amino acid sequence has an amino acid variant on position N29, and which substantially retains the biological activity of the peptide.

A further embodiment of the present invention relates to a composition comprising:
a. at least one cyclic peptide comprising an amino acid sequence of:
GLPVCGETCVGGTCNTPGCTCSWPVCTRN (SEQ ID NO: 1) or
GLPVCGETCFGGTCNTPGCSCTWPICTRD (SEQ ID NO: 2); and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5).

A further embodiment of the present invention relates to a composition comprising:
a. at least one cyclic peptide comprising an amino acid sequence of:
GLPVCGETCVGGTCNTPGCTCSWPVCTRN (SEQ ID NO: 1) or
GLPVCGETCFGGTCNTPGCSCTWPICTRD (SEQ ID NO: 2); and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5) or a variant of a peptide comprising an amino acid sequence of SEQ ID NO: 3, wherein the amino acid sequence has an amino acid variant on position N29, and which substantially retains the biological activity of the peptide.

Yet a further embodiment of the present invention relates to a composition comprising:
a. at least one cyclic peptide comprising an amino acid sequence of:
GLPVCGETCVGGTCNTPGCTCSWPVCTRN (SEQ ID NO: 1) or
GLPVCGETCFGGTCNTPGCSCTWPICTRD (SEQ ID NO: 2), or homologues thereof; and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5), or homologues thereof.

Yet a further embodiment of the present invention relates to a composition comprising:
a. at least one cyclic peptide comprising an amino acid sequence of:
GLPVCGETCVGGTCNTPGCTCSWPVCTRN (SEQ ID NO: 1) or
GLPVCGETCFGGTCNTPGCSCTWPICTRD (SEQ ID NO: 2), or homologues thereof; and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5), or homologues thereof, or a variant of a peptide comprising an amino acid sequence of SEQ ID NO: 3, wherein the amino acid sequence has an amino acid variant on position N29, and which substantially retains the biological activity of the peptide.

Another embodiment of the present invention relates to a composition comprising:
a. at least one cyclic peptide comprising an amino acid sequence of:
GLPVCGETCVGGTCNTPGCTCSWPVCTRN (SEQ ID NO: 1) or
GLPVCGETCFGGTCNTPGCSCTWPICTRD (SEQ ID NO: 2), or an amino acid sequence that is at least 65%, 70%, 75%, 80%, 85%, 90%, 91%, 92%, 93%, 94%, 95%, 96%, 97%, 98% or 99% identical to SEQ ID NO: 1 or SEQ ID NO: 2; and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5), or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5.

Another embodiment of the present invention relates to a composition comprising:
a. at least one cyclic peptide comprising an amino acid sequence of:
GLPVCGETCVGGTCNTPGCTCSWPVCTRN (SEQ ID NO: 1) or
GLPVCGETCFGGTCNTPGCSCTWPICTRD (SEQ ID NO: 2), or an amino acid sequence that is at least 65%, 70%, 75%, 80%, 85%, 90%, 91%, 92%, 93%, 94%, 95%, 96%, 97%, 98% or 99% identical to SEQ ID NO: 1 or SEQ ID NO: 2; and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5), or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. at least one cyclic peptide comprising an amino acid sequence of:
GLPVCGETCVGGTCNTPGCTCSWPVCTRN (SEQ ID NO: 1) or
GLPVCGETCFGGTCNTPGCSCTWPICTRD (SEQ ID NO: 2), or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 1 or SEQ ID NO: 2; and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5), or homologues thereof, or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5.

An embodiment of the present invention relates to a composition comprising:
a. at least one cyclic peptide comprising an amino acid sequence of:
GLPVCGETCVGGTCNTPGCTCSWPVCTRN (SEQ ID NO: 1) or
GLPVCGETCFGGTCNTPGCSCTWPICTRD (SEQ ID NO: 2), or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 1 or SEQ ID NO: 2; and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5), or homologues thereof, or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5, or a variant of a peptide comprising an amino acid sequence of SEQ ID NO: 3, wherein the amino acid sequence has an amino acid variant on position N29, and which substantially retains the biological activity of the peptide.

A further embodiment of the present invention relates to a composition comprising:
a. at least one cyclic peptide comprising an amino acid sequence of:
GLPVCGETCVGGTCNTPGCTCSWPVCTRN (SEQ ID NO: 1) or
GLPVCGETCVGGTCNTPGCTCSWPKCTRN (SEQ ID NO: 6); and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5).

A further embodiment of the present invention relates to a composition comprising:
a. at least one cyclic peptide comprising an amino acid sequence of:
GLPVCGETCVGGTCNTPGCTCSWPVCTRN (SEQ ID NO: 1) or
GLPVCGETCVGGTCNTPGCTCSWPKCTRN (SEQ ID NO: 6); and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5) or a variant of a peptide comprising an amino acid sequence of SEQ ID NO: 3, wherein the amino acid sequence has an amino acid variant on position N29, and which substantially retains the biological activity of the peptide.

Yet a further embodiment of the present invention relates to a composition comprising:
a. at least one cyclic peptide comprising an amino acid sequence of:
GLPVCGETCVGGTCNTPGCTCSWPVCTRN (SEQ ID NO: 1) or
GLPVCGETCVGGTCNTPGCTCSWPKCTRN (SEQ ID NO: 6), or homologues thereof; and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5), or homologues thereof.

Yet a further embodiment of the present invention relates to a composition comprising:
a. at least one cyclic peptide comprising an amino acid sequence of:
GLPVCGETCVGGTCNTPGCTCSWPVCTRN (SEQ ID NO: 1) or
GLPVCGETCVGGTCNTPGCTCSWPKCTRN (SEQ ID NO: 6), or homologues thereof; and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5), or homologues thereof, or a variant of a peptide comprising an amino acid sequence of SEQ ID NO: 3, wherein the amino acid sequence has an amino acid variant on position N29, and which substantially retains the biological activity of the peptide.

Another embodiment of the present invention relates to a composition comprising:
a. at least one cyclic peptide comprising an amino acid sequence of:
GLPVCGETCVGGTCNTPGCTCSWPVCTRN (SEQ ID NO: 1) or
GLPVCGETCVGGTCNTPGCTCSWPKCTRN (SEQ ID NO: 6), or an amino acid sequence that is at least 65%, 70%, 75%, 80%, 85%, 90%, 91%, 92%, 93%, 94%, 95%, 96%, 97%, 98% or 99% identical to SEQ ID NO: 1 or SEQ ID NO: 6; and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5), or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5.

Another embodiment of the present invention relates to a composition comprising:
a. at least one cyclic peptide comprising an amino acid sequence of:
GLPVCGETCVGGTCNTPGCTCSWPVCTRN (SEQ ID NO: 1) or
GLPVCGETCVGGTCNTPGCTCSWPKCTRN (SEQ ID NO: 6), or an amino acid sequence that is at least 65%, 70%, 75%, 80%, 85%, 90%, 91%, 92%, 93%, 94%, 95%, 96%, 97%, 98% or 99% identical to SEQ ID NO: 1 or SEQ ID NO: 6; and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5), or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. at least one cyclic peptide comprising an amino acid sequence of:
GLPVCGETCVGGTCNTPGCTCSWPVCTRN (SEQ ID NO: 1) or
GLPVCGETCVGGTCNTPGCTCSWPKCTRN (SEQ ID NO: 6), or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 1 or SEQ ID NO: 6; and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5), or homologues thereof, or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5.

An embodiment of the present invention relates to a composition comprising:
a. at least one cyclic peptide comprising an amino acid sequence of:
GLPVCGETCVGGTCNTPGCTCSWPVCTRN (SEQ ID NO: 1) or
GLPVCGETCVGGTCNTPGCTCSWPKCTRN (SEQ ID NO: 6), or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 1 or SEQ ID NO: 6; and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5), or homologues thereof, or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5, or a variant of a peptide comprising an amino acid sequence of SEQ ID NO: 3, wherein the amino acid sequence has an amino acid variant on position N29, and which substantially retains the biological activity of the peptide.

A further embodiment of the present invention relates to a composition comprising:
a. at least one cyclic peptide comprising an amino acid sequence of:
GLPVCGETCVGGTCNTPGCTCSWPKCTRN (SEQ ID NO: 6) or
GLPVCGETCFGGTCNTPGCSCTWPICTRD (SEQ ID NO: 2); and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5).

A further embodiment of the present invention relates to a composition comprising:
a. at least one cyclic peptide comprising an amino acid sequence of:
GLPVCGETCVGGTCNTPGCTCSWPKCTRN (SEQ ID NO: 6) or
GLPVCGETCFGGTCNTPGCSCTWPICTRD (SEQ ID NO: 2); and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5) or a variant of a peptide comprising an amino acid sequence of SEQ ID NO: 3, wherein the amino acid sequence has an amino acid variant on position N29, and which substantially retains the biological activity of the peptide.

Yet a further embodiment of the present invention relates to a composition comprising:
a. at least one cyclic peptide comprising an amino acid sequence of:
GLPVCGETCVGGTCNTPGCTCSWPKCTRN (SEQ ID NO: 6) or
GLPVCGETCFGGTCNTPGCSCTWPICTRD (SEQ ID NO: 2), or homologues thereof; and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5), or homologues thereof.

Yet a further embodiment of the present invention relates to a composition comprising:
a. at least one cyclic peptide comprising an amino acid sequence of:
GLPVCGETCVGGTCNTPGCTCSWPKCTRN (SEQ ID NO: 6) or
GLPVCGETCFGGTCNTPGCSCTWPICTRD (SEQ ID NO: 2), or homologues thereof; and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5), or homologues thereof, or a variant of a peptide comprising an amino acid sequence of SEQ ID NO: 3, wherein the amino acid sequence has an amino acid variant on position N29, and which substantially retains the biological activity of the peptide.

Another embodiment of the present invention relates to a composition comprising:
a. at least one cyclic peptide comprising an amino acid sequence of:
GLPVCGETCVGGTCNTPGCTCSWPKCTRN (SEQ ID NO: 6) or
GLPVCGETCFGGTCNTPGCSCTWPICTRD (SEQ ID NO: 2), or an amino acid sequence that is at least 65%, 70%, 75%, 80%, 85%, 90%, 91%, 92%, 93%, 94%, 95%, 96%, 97%, 98% or 99% identical to SEQ ID NO: 6 or SEQ ID NO: 2; and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5), or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5.

Another embodiment of the present invention relates to a composition comprising:
a. at least one cyclic peptide comprising an amino acid sequence of:
GLPVCGETCVGGTCNTPGCTCSWPKCTRN (SEQ ID NO: 6) or
GLPVCGETCFGGTCNTPGCSCTWPICTRD (SEQ ID NO: 2), or an amino acid sequence that is at least 65%, 70%, 75%, 80%, 85%, 90%, 91%, 92%, 93%, 94%, 95%, 96%, 97%, 98% or 99% identical to SEQ ID NO: 6 or SEQ ID NO: 2; and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5), or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. at least one cyclic peptide comprising an amino acid sequence of:
GLPVCGETCVGGTCNTPGCTCSWPKCTRN (SEQ ID NO: 6) or
GLPVCGETCFGGTCNTPGCSCTWPICTRD (SEQ ID NO: 2), or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 6 or SEQ ID NO: 2; and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5), or homologues thereof, or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5.

An embodiment of the present invention relates to a composition comprising:
a. at least one cyclic peptide comprising an amino acid sequence of:
GLPVCGETCVGGTCNTPGCTCSWPKCTRN (SEQ ID NO: 6) or
GLPVCGETCFGGTCNTPGCSCTWPICTRD (SEQ ID NO: 2), or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 6 or SEQ ID NO: 2; and
b. at least one second peptide comprising an amino acid sequence of any of
SPTCIPSGQPCPYNENCCSQSCTFKENENGNTVKRCD (SEQ ID NO: 3),
RSCCPCYWGGCPWGQNCYPEGCSGPKV (SEQ ID NO: 4) or
GSQYCVPVDQPCSLNTQPCCDDATCTQERNENGHTVYYCRA (SEQ ID NO: 5), or homologues thereof, or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 1; and
b. at least one second peptide comprising an amino acid sequence of any of SEQ ID NOs: 3 to 5.

An embodiment of the present invention relates to a composition comprising:
a. a cyclic peptide comprising an amino acid sequence of: SEQ ID NO: 1; and
b. at least one second peptide comprising an amino acid sequence of any of SEQ ID NOs: 3 to 5, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 1, or homologues thereof; and
b. at least one second peptide comprising an amino acid sequence of any of SEQ ID NOs: 3 to 5, or homologues thereof.

An embodiment of the present invention relates to a composition comprising:
a. a cyclic peptide comprising an amino acid sequence of: SEQ ID NO: 1, or homologues thereof; and
b. at least one second peptide comprising an amino acid sequence of any of SEQ ID NOs: 3 to 5, or homologues thereof, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 1, or an amino acid sequence that is at least 65%, 70%, 75%, 80%, 85%, 90%, 91%, 92%, 93%, 94%, 95%, 96%, 97%, 98% or 99% identical to SEQ ID NO: 1; and
b. at least one second peptide comprising an amino acid sequence of any of SEQ ID NOs: 3 to 5, or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5.

An embodiment of the present invention relates to a composition comprising:
a. a cyclic peptide comprising an amino acid sequence of: SEQ ID NO: 1, or an amino acid sequence that is at least 65%, 70%, 75%, 80%, 85%, 90%, 91%, 92%, 93%, 94%, 95%, 96%, 97%, 98% or 99% identical to SEQ ID NO: 1; and
b. at least one second peptide comprising an amino acid sequence of any of SEQ ID NOs: 3 to 5, or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 1, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 1; and
b. at least one second peptide comprising an amino acid sequence of any of SEQ ID NOs: 3 to 5, or homologues thereof, or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5.

An embodiment of the present invention relates to a composition comprising:
a. a cyclic peptide comprising an amino acid sequence of: SEQ ID NO: 1, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 1; and
b. at least one second peptide comprising an amino acid sequence of any of SEQ ID NOs: 3 to 5, or homologues thereof, or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 2; and
b. at least one second peptide comprising an amino acid sequence of any of SEQ ID NOs: 3 to 5.

An embodiment of the present invention relates to a composition comprising:
a. a cyclic peptide comprising an amino acid sequence of: SEQ ID NO: 2; and
b. at least one second peptide comprising an amino acid sequence of any of SEQ ID NOs: 3 to 5, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 2, or homologues thereof; and
b. at least one second peptide comprising an amino acid sequence of any of SEQ ID NOs: 3 to 5, or homologues thereof.

An embodiment of the present invention relates to a composition comprising:
a. a cyclic peptide comprising an amino acid sequence of: SEQ ID NO: 2, or homologues thereof; and
b. at least one second peptide comprising an amino acid sequence of any of SEQ ID NOs: 3 to 5, or homologues thereof, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 2, or an amino acid sequence that is at least 65%, 70%, 75%, 80%, 85%, 90%, 91%, 92%, 93%, 94%, 95%, 96%, 97%, 98% or 99% identical to SEQ ID NO: 2; and
b. at least one second peptide comprising an amino acid sequence of any of SEQ ID NOs: 3 to 5, or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5.

An embodiment of the present invention relates to a composition comprising:
a. a cyclic peptide comprising an amino acid sequence of: SEQ ID NO: 2, or an amino acid sequence that is at least 65%, 70%, 75%, 80%, 85%, 90%, 91%, 92%, 93%, 94%, 95%, 96%, 97%, 98% or 99% identical to SEQ ID NO: 2; and
b. at least one second peptide comprising an amino acid sequence of any of SEQ ID NOs: 3 to 5, or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 2, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 2; and
b. at least one second peptide comprising an amino acid sequence of any of SEQ ID NOs: 3 to 5, or homologues thereof, or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5.

An embodiment of the present invention relates to a composition comprising:
a. a cyclic peptide comprising an amino acid sequence of: SEQ ID NO: 2, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 2; and
b. at least one second peptide comprising an amino acid sequence of any of SEQ ID NOs: 3 to 5, or homologues thereof, or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 6; and
b. at least one second peptide comprising an amino acid sequence of any of SEQ ID NOs: 3 to 5.

An embodiment of the present invention relates to a composition comprising:
a. a cyclic peptide comprising an amino acid sequence of: SEQ ID NO: 6; and
b. at least one second peptide comprising an amino acid sequence of any of SEQ ID NOs: 3 to 5, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 6, or homologues thereof; and
b. at least one second peptide comprising an amino acid sequence of any of SEQ ID NOs: 3 to 5, or homologues thereof.

An embodiment of the present invention relates to a composition comprising:
a. a cyclic peptide comprising an amino acid sequence of: SEQ ID NO: 6, or homologues thereof; and
b. at least one second peptide comprising an amino acid sequence of any of SEQ ID NOs: 3 to 5, or homologues thereof, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 6, or an amino acid sequence that is at least 65%, 70%, 75%, 80%, 85%, 90%, 91%, 92%, 93%, 94%, 95%, 96%, 97%, 98% or 99% identical to SEQ ID NO: 6; and
b. at least one second peptide comprising an amino acid sequence of any of SEQ ID NOs: 3 to 5, or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5.

An embodiment of the present invention relates to a composition comprising:
a. a cyclic peptide comprising an amino acid sequence of: SEQ ID NO: 6, or an amino acid sequence that is at least 65%, 70%, 75%, 80%, 85%, 90%, 91%, 92%, 93%, 94%, 95%, 96%, 97%, 98% or 99% identical to SEQ ID NO: 6; and
b. at least one second peptide comprising an amino acid sequence of any of SEQ ID NOs: 3 to 5, or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 6, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 6; and
b. at least one second peptide comprising an amino acid sequence of any of SEQ ID NOs: 3 to 5, or homologues thereof, or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5.

An embodiment of the present invention relates to a composition comprising:
a. a cyclic peptide comprising an amino acid sequence of: SEQ ID NO: 6, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 6; and
b. at least one second peptide comprising an amino acid sequence of any of SEQ ID NOs: 3 to 5, or homologues thereof, or an amino acid sequence that is at least 65%, 70%, 75%, 80%, 85%, 90%, 91%, 92%, 93%, 94%, 95%, 96%, 97%, 98% or 99% identical to any of SEQ ID NOs: 3 to 5, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 1; and
b. at least one second peptide comprising an amino acid sequence of SEQ ID NOs: 3 or 4.

An embodiment of the present invention relates to a composition comprising:
a. a cyclic peptide comprising an amino acid sequence of: SEQ ID NO: 1; and
b. at least one second peptide comprising an amino acid sequence of SEQ ID NOs: 3 or 4, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 1, or homologues thereof; and
b. at least one second peptide comprising an amino acid sequence of SEQ ID NOs: 3 or 4, or homologues thereof.

An embodiment of the present invention relates to a composition comprising:
a. a cyclic peptide comprising an amino acid sequence of: SEQ ID NO: 1, or homologues thereof; and
b. at least one second peptide comprising an amino acid sequence of SEQ ID NOs: 3 or 4, or homologues thereof, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 1, or an amino acid sequence that is at least 65%, 70%, 75%, 80%, 85%, 90%, 91%, 92%, 93%, 94%, 95%, 96%, 97%, 98% or 99% identical to SEQ ID NO: 1; and
b. at least one second peptide comprising an amino acid sequence of SEQ ID NOs: 3 or 4, or an amino acid sequence that is at least 65%, 70%, 75%, 80%, 85%, 90%, 91%, 92%, 93%, 94%, 95%, 96%, 97%, 98% or 99% identical to SEQ ID NOs: 3 or 4.

An embodiment of the present invention relates to a composition comprising:
a. a cyclic peptide comprising an amino acid sequence of: SEQ ID NO: 1, or an amino acid sequence that is at least 65%, 70%, 75%, 80%, 85%, 90%, 91%, 92%, 93%, 94%, 95%, 96%, 97%, 98% or 99% identical to SEQ ID NO: 1; and
b. at least one second peptide comprising an amino acid sequence of SEQ ID NOs: 3 or 4, or an amino acid sequence that is at least 65%, 70%, 75%, 80%, 85%, 90%, 91%, 92%, 93%, 94%, 95%, 96%, 97%, 98% or 99% identical to SEQ ID NOs: 3 or 4, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 1, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 1; and
b. at least one second peptide comprising an amino acid sequence of SEQ ID NOs: 3 or 4, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NOs: 3 or 4.

An embodiment of the present invention relates to a composition comprising:
a. a cyclic peptide comprising an amino acid sequence of: SEQ ID NO: 1, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 1; and
b. at least one second peptide comprising an amino acid sequence of SEQ ID NOs: 3 or 4, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NOs: 3 or 4, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 1; and
b. at least one second peptide comprising an amino acid sequence of SEQ ID NOs: 3 or 5.

An embodiment of the present invention relates to a composition comprising:
a. a cyclic peptide comprising an amino acid sequence of: SEQ ID NO: 1; and
b. at least one second peptide comprising an amino acid sequence of SEQ ID NOs: 3 or 5, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 1, or homologues thereof; and
b. at least one second peptide comprising an amino acid sequence of SEQ ID NOs: 3 or 5, or homologues thereof.

An embodiment of the present invention relates to a composition comprising:
a. a cyclic peptide comprising an amino acid sequence of: SEQ ID NO: 1, or homologues thereof; and
b. at least one second peptide comprising an amino acid sequence of SEQ ID NOs: 3 or 5, or homologues thereof, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 1, or an amino acid sequence that is at least 65%, 70%, 75%, 80%, 85%, 90%, 91%, 92%, 93%, 94%, 95%, 96%, 97%, 98% or 99% identical to SEQ ID NO: 1; and
b. at least one second peptide comprising an amino acid sequence of SEQ ID NOs: 3 or 5, or an amino acid sequence that is at least 65%, 70%, 75%, 80%, 85%, 90%, 91%, 92%, 93%, 94%, 95%, 96%, 97%, 98% or 99% identical to SEQ ID NOs: 3 or 5.

An embodiment of the present invention relates to a composition comprising:
a. a cyclic peptide comprising an amino acid sequence of: SEQ ID NO: 1, or an amino acid sequence that is at least 65%, 70%, 75%, 80%, 85%, 90%, 91%, 92%, 93%, 94%, 95%, 96%, 97%, 98% or 99% identical to SEQ ID NO: 1; and
b. at least one second peptide comprising an amino acid sequence of SEQ ID NOs: 3 or 5, or an amino acid sequence that is at least 65%, 70%, 75%, 80%, 85%, 90%, 91%, 92%, 93%, 94%, 95%, 96%, 97%, 98% or 99% identical to SEQ ID NOs: 3 or 5, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 1, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 1; and
b. at least one second peptide comprising an amino acid sequence of SEQ ID NOs: 3 or 5, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NOs: 3 or 5.

An embodiment of the present invention relates to a composition comprising:
a. a cyclic peptide comprising an amino acid sequence of: SEQ ID NO: 1, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 1; and
b. at least one second peptide comprising an amino acid sequence of SEQ ID NOs: 3 or 5, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NOs: 3 or 5, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 1; and
b. at least one second peptide comprising an amino acid sequence of SEQ ID NOs: 4 or 5.

An embodiment of the present invention relates to a composition comprising:
a. a cyclic peptide comprising an amino acid sequence of: SEQ ID NO: 1, or homologues thereof; and
b. at least one second peptide comprising an amino acid sequence of SEQ ID NOs: 4 or 5, or homologues thereof.

An embodiment of the present invention relates to a composition comprising:
a. a cyclic peptide comprising an amino acid sequence of: SEQ ID NO: 1, or an amino acid sequence that is at least 65%, 70%, 75%, 80%, 85%, 90%, 91%, 92%, 93%, 94%, 95%, 96%, 97%, 98% or 99% identical to SEQ ID NO: 1; and
b. at least one second peptide comprising an amino acid sequence of SEQ ID NOs: 4 or 5, or an amino acid sequence that is at least 65%, 70%, 75%, 80%, 85%, 90%, 91%, 92%, 93%, 94%, 95%, 96%, 97%, 98% or 99% identical to SEQ ID NOs: 4 or 5.

An embodiment of the present invention relates to a composition comprising:
a. a cyclic peptide comprising an amino acid sequence of: SEQ ID NO: 1, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 1; and
b. at least one second peptide comprising an amino acid sequence of SEQ ID NOs: 4 or 5, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NOs: 4 or 5.

An embodiment of the present invention relates to a composition comprising:
a. a cyclic peptide comprising an amino acid sequence of: SEQ ID NO: 2; and
b. at least one second peptide comprising an amino acid sequence of SEQ ID NOs: 3 or 4.

An embodiment of the present invention relates to a composition comprising:
a. a cyclic peptide comprising an amino acid sequence of: SEQ ID NO: 2; and
b. at least one second peptide comprising an amino acid sequence of SEQ ID NOs: 3 or 4, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 2, or homologues thereof; and
b. at least one second peptide comprising an amino acid sequence of SEQ ID NOs: 3 or 4, or homologues thereof.

An embodiment of the present invention relates to a composition comprising:
a. a cyclic peptide comprising an amino acid sequence of: SEQ ID NO: 2, or homologues thereof; and
b. at least one second peptide comprising an amino acid sequence of SEQ ID NOs: 3 or 4, or homologues thereof, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 2 or an amino acid sequence that is at least 65%, 70%, 75%, 80%, 85%, 90%, 91%, 92%, 93%, 94%, 95%, 96%, 97%, 98% or 99% identical to SEQ ID NO: 2; and
b. at least one second peptide comprising an amino acid sequence of SEQ ID NOs: 3 or 4 or an amino acid sequence that is at least 65%, 70%, 75%, 80%, 85%, 90%, 91%, 92%, 93%, 94%, 95%, 96%, 97%, 98% or 99% identical to SEQ ID NOs: 3 or 4.

An embodiment of the present invention relates to a composition comprising:
a. a cyclic peptide comprising an amino acid sequence of: SEQ ID NO: 2 or an amino acid sequence that is at least 65%, 70%, 75%, 80%, 85%, 90%, 91%, 92%, 93%, 94%, 95%, 96%, 97%, 98% or 99% identical to SEQ ID NO: 2; and
b. at least one second peptide comprising an amino acid sequence of SEQ ID NOs: 3 or 4 or an amino acid sequence that is at least 65%, 70%, 75%, 80%, 85%, 90%, 91%, 92%, 93%, 94%, 95%, 96%, 97%, 98% or 99% identical to SEQ ID NOs: 3 or 4, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 2, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 2; and
b. at least one second peptide comprising an amino acid sequence of SEQ ID NOs: 3 or 4, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NOs: 3 or 4.

An embodiment of the present invention relates to a composition comprising:
a. a cyclic peptide comprising an amino acid sequence of: SEQ ID NO: 2, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 2; and
b. at least one second peptide comprising an amino acid sequence of SEQ ID NOs: 3 or 4, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NOs: 3 or 4, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 2; and
b. at least one second peptide comprising an amino acid sequence of SEQ ID NOs: 3 or 5.

An embodiment of the present invention relates to a composition comprising:
a. a cyclic peptide comprising an amino acid sequence of: SEQ ID NO: 2; and
b. at least one second peptide comprising an amino acid sequence of SEQ ID NOs: 3 or 5, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 2, or homologues thereof; and
b. at least one second peptide comprising an amino acid sequence of SEQ ID NOs: 3 or 5, or homologues thereof.

An embodiment of the present invention relates to a composition comprising:
a. a cyclic peptide comprising an amino acid sequence of: SEQ ID NO: 2, or homologues thereof; and
b. at least one second peptide comprising an amino acid sequence of SEQ ID NOs: 3 or 5, or homologues thereof, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 2 or an amino acid sequence that is at least 65%, 70%, 75%, 80%, 85%, 90%, 91%, 92%, 93%, 94%, 95%, 96%, 97%, 98% or 99% identical to SEQ ID NO: 2; and
b. at least one second peptide comprising an amino acid sequence of SEQ ID NOs: 3 or 5 or an amino acid sequence that is at least 65%, 70%, 75%, 80%, 85%, 90%, 91%, 92%, 93%, 94%, 95%, 96%, 97%, 98% or 99% identical to SEQ ID NOs: 3 or 5.

An embodiment of the present invention relates to a composition comprising:
a. a cyclic peptide comprising an amino acid sequence of: SEQ ID NO: 2 or an amino acid sequence that is at least 65%, 70%, 75%, 80%, 85%, 90%, 91%, 92%, 93%, 94%, 95%, 96%, 97%, 98% or 99% identical to SEQ ID NO: 2; and
b. at least one second peptide comprising an amino acid sequence of SEQ ID NOs: 3 or 5 or an amino acid sequence that is at least 65%, 70%, 75%, 80%, 85%, 90%, 91%, 92%, 93%, 94%, 95%, 96%, 97%, 98% or 99% identical to SEQ ID NOs: 3 or 5, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 2, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 2; and
b. at least one second peptide comprising an amino acid sequence of SEQ ID NOs: 3 or 5, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NOs: 3 or 5.

An embodiment of the present invention relates to a composition comprising:
a. a cyclic peptide comprising an amino acid sequence of: SEQ ID NO: 2, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 2; and
b. at least one second peptide comprising an amino acid sequence of SEQ ID NOs: 3 or 5, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NOs: 3 or 5, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 2; and
b. at least one second peptide comprising an amino acid sequence of SEQ ID NOs: 4 or 5.

An embodiment of the present invention relates to a composition comprising:
a. a cyclic peptide comprising an amino acid sequence of: SEQ ID NO: 2, or homologues thereof; and
b. at least one second peptide comprising an amino acid sequence of SEQ ID NOs: 4 or 5, or homologues thereof.

An embodiment of the present invention relates to a composition comprising:
a. a cyclic peptide comprising an amino acid sequence of: SEQ ID NO: 2 or an amino acid sequence that is at least 65%, 70%, 75%, 80%, 85%, 90%, 91%, 92%, 93%, 94%, 95%, 96%, 97%, 98% or 99% identical to SEQ ID NO: 2; and
b. at least one second peptide comprising an amino acid sequence of SEQ ID NOs: 4 or 5 or an amino acid sequence that is at least 65%, 70%, 75%, 80%, 85%, 90%, 91%, 92%, 93%, 94%, 95%, 96%, 97%, 98% or 99% identical to SEQ ID NOs: 4 or 5.

An embodiment of the present invention relates to a composition comprising:
a. a cyclic peptide comprising an amino acid sequence of: SEQ ID NO: 2, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 2; and
b. at least one second peptide comprising an amino acid sequence of SEQ ID NOs: 4 or 5, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NOs: 4 or 5.

An embodiment of the present invention relates to a composition comprising:
a. a cyclic peptide comprising an amino acid sequence of: SEQ ID NO: 6; and
b. at least one second peptide comprising an amino acid sequence of SEQ ID NOs: 3 or 4.

An embodiment of the present invention relates to a composition comprising:
a. a cyclic peptide comprising an amino acid sequence of: SEQ ID NO: 6; and
b. at least one second peptide comprising an amino acid sequence of SEQ ID NOs: 3 or 4, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 6, or homologues thereof; and
b. at least one second peptide comprising an amino acid sequence of SEQ ID NOs: 3 or 4, or homologues thereof.

An embodiment of the present invention relates to a composition comprising:
a. a cyclic peptide comprising an amino acid sequence of: SEQ ID NO: 6, or homologues thereof; and
b. at least one second peptide comprising an amino acid sequence of SEQ ID NOs: 3 or 4, or homologues thereof, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 6 or an amino acid sequence that is at least 65%, 70%, 75%, 80%, 85%, 90%, 91%, 92%, 93%, 94%, 95%, 96%, 97%, 98% or 99% identical to SEQ ID NO: 6; and
b. at least one second peptide comprising an amino acid sequence of SEQ ID NOs: 3 or 4 or an amino acid sequence that is at least 65%, 70%, 75%, 80%, 85%, 90%, 91%, 92%, 93%, 94%, 95%, 96%, 97%, 98% or 99% identical to SEQ ID NOs: 3 or 4.

An embodiment of the present invention relates to a composition comprising:
a. a cyclic peptide comprising an amino acid sequence of: SEQ ID NO: 6 or an amino acid sequence that is at least 65%, 70%, 75%, 80%, 85%, 90%, 91%, 92%, 93%, 94%, 95%, 96%, 97%, 98% or 99% identical to SEQ ID NO: 6; and
b. at least one second peptide comprising an amino acid sequence of SEQ ID NOs: 3 or 4 or an amino acid sequence that is at least 65%, 70%, 75%, 80%, 85%, 90%, 91%, 92%, 93%, 94%, 95%, 96%, 97%, 98% or 99% identical to SEQ ID NOs: 3 or 4, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 6, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 6; and
b. at least one second peptide comprising an amino acid sequence of SEQ ID NOs: 3 or 4, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NOs: 3 or 4.

An embodiment of the present invention relates to a composition comprising:
a. a cyclic peptide comprising an amino acid sequence of: SEQ ID NO: 6, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 6; and
b. at least one second peptide comprising an amino acid sequence of SEQ ID NOs: 3 or 4, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NOs: 3 or 4, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 6; and
b. at least one second peptide comprising an amino acid sequence of SEQ ID NOs: 3 or 5.

An embodiment of the present invention relates to a composition comprising:
a. a cyclic peptide comprising an amino acid sequence of: SEQ ID NO: 6; and
b. at least one second peptide comprising an amino acid sequence of SEQ ID NOs: 3 or 5, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 6, or homologues thereof; and
b. at least one second peptide comprising an amino acid sequence of SEQ ID NOs: 3 or 5, or homologues thereof.

An embodiment of the present invention relates to a composition comprising:
a. a cyclic peptide comprising an amino acid sequence of: SEQ ID NO: 6, or homologues thereof; and
b. at least one second peptide comprising an amino acid sequence of SEQ ID NOs: 3 or 5, or homologues thereof, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 6 or an amino acid sequence that is at least 65%, 70%, 75%, 80%, 85%, 90%, 91%, 92%, 93%, 94%, 95%, 96%, 97%, 98% or 99% identical to SEQ ID NO: 6; and
b. at least one second peptide comprising an amino acid sequence of SEQ ID NOs: 3 or 5 or an amino acid sequence that is at least 65%, 70%, 75%, 80%, 85%, 90%, 91%, 92%, 93%, 94%, 95%, 96%, 97%, 98% or 99% identical to SEQ ID NOs: 3 or 5.

An embodiment of the present invention relates to a composition comprising:
a. a cyclic peptide comprising an amino acid sequence of: SEQ ID NO: 6 or an amino acid sequence that is at least 65%, 70%, 75%, 80%, 85%, 90%, 91%, 92%, 93%, 94%, 95%, 96%, 97%, 98% or 99% identical to SEQ ID NO: 6; and
b. at least one second peptide comprising an amino acid sequence of SEQ ID NOs: 3 or 5 or an amino acid sequence that is at least 65%, 70%, 75%, 80%, 85%, 90%, 91%, 92%, 93%, 94%, 95%, 96%, 97%, 98% or 99% identical to SEQ ID NOs: 3 or 5, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 6, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 6; and
b. at least one second peptide comprising an amino acid sequence of SEQ ID NOs: 3 or 5, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NOs: 3 or 5.

An embodiment of the present invention relates to a composition comprising:
a. a cyclic peptide comprising an amino acid sequence of: SEQ ID NO: 6, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 6; and
b. at least one second peptide comprising an amino acid sequence of SEQ ID NOs: 3 or 5, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NOs: 3 or 5, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 6; and
b. at least one second peptide comprising an amino acid sequence of SEQ ID NOs: 4 or 5.

An embodiment of the present invention relates to a composition comprising:
a. a cyclic peptide comprising an amino acid sequence of: SEQ ID NO: 6, or homologues thereof; and
b. at least one second peptide comprising an amino acid sequence of SEQ ID NOs: 4 or 5, or homologues thereof.

An embodiment of the present invention relates to a composition comprising:
a. a cyclic peptide comprising an amino acid sequence of: SEQ ID NO: 6 or an amino acid sequence that is at least 65%, 70%, 75%, 80%, 85%, 90%, 91%, 92%, 93%, 94%, 95%, 96%, 97%, 98% or 99% identical to SEQ ID NO: 6; and
b. at least one second peptide comprising an amino acid sequence of SEQ ID NOs: 4 or 5 or an amino acid sequence that is at least 65%, 70%, 75%, 80%, 85%, 90%, 91%, 92%, 93%, 94%, 95%, 96%, 97%, 98% or 99% identical to SEQ ID NOs: 4 or 5.

An embodiment of the present invention relates to a composition comprising:
a. a cyclic peptide comprising an amino acid sequence of: SEQ ID NO: 6, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 6; and
b. at least one second peptide comprising an amino acid sequence of SEQ ID NOs: 4 or 5, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NOs: 4 or 5.

An embodiment of the present invention relates to a composition comprising:
a. a cyclic peptide comprising an amino acid sequence of: SEQ ID NO: 1; and
b. a second peptide comprising an amino acid sequence of: SEQ ID NO: 3.

An embodiment of the present invention relates to a composition comprising:
a. a cyclic peptide comprising an amino acid sequence of: SEQ ID NO: 1; and
b. a second peptide comprising an amino acid sequence of: SEQ ID NO: 3, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 1, or homologues thereof; and
b. a second peptide comprising an amino acid sequence of: SEQ ID NO: 3, or homologues thereof.

An embodiment of the present invention relates to a composition comprising:
a. a cyclic peptide comprising an amino acid sequence of: SEQ ID NO: 1, or homologues thereof; and
b. a second peptide comprising an amino acid sequence of: SEQ ID NO: 3, or homologues thereof, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 1, or an amino acid sequence that is at least 65%, 70%, 75%, 80%, 85%, 90%, 91%, 92%, 93%, 94%, 95%, 96%, 97%, 98% or 99% identical to SEQ ID NO: 1; and
b. a second peptide comprising an amino acid sequence of: SEQ ID NO: 3 or an amino acid sequence that is at least 65%, 70%, 75%, 80%, 85%, 90%, 91%, 92%, 93%, 94%, 95%, 96%, 97%, 98% or 99% identical to SEQ ID NO: 3.

An embodiment of the present invention relates to a composition comprising:
a. a cyclic peptide comprising an amino acid sequence of: SEQ ID NO: 1, or an amino acid sequence that is at least 65%, 70%, 75%, 80%, 85%, 90%, 91%, 92%, 93%, 94%, 95%, 96%, 97%, 98% or 99% identical to SEQ ID NO: 1; and
b. a second peptide comprising an amino acid sequence of: SEQ ID NO: 3 or an amino acid sequence that is at least 65%, 70%, 75%, 80%, 85%, 90%, 91%, 92%, 93%, 94%, 95%, 96%, 97%, 98% or 99% identical to SEQ ID NO: 3, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 1, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 1; and
b. a second peptide comprising an amino acid sequence of: SEQ ID NO: 3, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 3.

An embodiment of the present invention relates to a composition comprising:
a. a cyclic peptide comprising an amino acid sequence of: SEQ ID NO: 1, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 1; and
b. a second peptide comprising an amino acid sequence of: SEQ ID NO: 3, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 3, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 2; and
b. a second peptide comprising an amino acid sequence of: SEQ ID NO: 3.

An embodiment of the present invention relates to a composition comprising:
a. a cyclic peptide comprising an amino acid sequence of: SEQ ID NO: 2; and
b. a second peptide comprising an amino acid sequence of: SEQ ID NO: 3, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 2, or homologues thereof; and
b. a second peptide comprising an amino acid sequence of: SEQ ID NO: 3, or homologues thereof.

An embodiment of the present invention relates to a composition comprising:
a. a cyclic peptide comprising an amino acid sequence of: SEQ ID NO: 2, or homologues thereof; and
b. a second peptide comprising an amino acid sequence of: SEQ ID NO: 3, or homologues thereof, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 2 or an amino acid sequence that is at least 65%, 70%, 75%, 80%, 85%, 90%, 91%, 92%, 93%, 94%, 95%, 96%, 97%, 98% or 99% identical to SEQ ID NO: 2; and
b. a second peptide comprising an amino acid sequence of: SEQ ID NO: 3 or an amino acid sequence that is at least 65%, 70%, 75%, 80%, 85%, 90%, 91%, 92%, 93%, 94%, 95%, 96%, 97%, 98% or 99% identical to SEQ ID NO: 3.

An embodiment of the present invention relates to a composition comprising:
a. a cyclic peptide comprising an amino acid sequence of: SEQ ID NO: 2 or an amino acid sequence that is at least 65%, 70%, 75%, 80%, 85%, 90%, 91%, 92%, 93%, 94%, 95%, 96%, 97%, 98% or 99% identical to SEQ ID NO: 2; and
b. a second peptide comprising an amino acid sequence of: SEQ ID NO: 3 or an amino acid sequence that is at least 65%, 70%, 75%, 80%, 85%, 90%, 91%, 92%, 93%, 94%, 95%, 96%, 97%, 98% or 99% identical to SEQ ID NO: 3, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 2, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 2; and
b. a second peptide comprising an amino acid sequence of: SEQ ID NO: 3, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 3.

An embodiment of the present invention relates to a composition comprising:
a. a cyclic peptide comprising an amino acid sequence of: SEQ ID NO: 2, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 2; and
b. a second peptide comprising an amino acid sequence of: SEQ ID NO: 3, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 3, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 6; and
b. a second peptide comprising an amino acid sequence of: SEQ ID NO: 3.

An embodiment of the present invention relates to a composition comprising:
a. a cyclic peptide comprising an amino acid sequence of: SEQ ID NO: 6; and
b. a second peptide comprising an amino acid sequence of: SEQ ID NO: 3, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 6, or homologues thereof; and
b. a second peptide comprising an amino acid sequence of: SEQ ID NO: 3, or homologues thereof.

An embodiment of the present invention relates to a composition comprising:
a. a cyclic peptide comprising an amino acid sequence of: SEQ ID NO: 6, or homologues thereof; and
b. a second peptide comprising an amino acid sequence of: SEQ ID NO: 3, or homologues thereof, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 6 or an amino acid sequence that is at least 65%, 70%, 75%, 80%, 85%, 90%, 91%, 92%, 93%, 94%, 95%, 96%, 97%, 98% or 99% identical to SEQ ID NO: 6; and
b. a second peptide comprising an amino acid sequence of: SEQ ID NO: 3 or an amino acid sequence that is at least 65%, 70%, 75%, 80%, 85%, 90%, 91%, 92%, 93%, 94%, 95%, 96%, 97%, 98% or 99% identical to SEQ ID NO: 3.

An embodiment of the present invention relates to a composition comprising:
a. a cyclic peptide comprising an amino acid sequence of: SEQ ID NO: 6 or an amino acid sequence that is at least 65%, 70%, 75%, 80%, 85%, 90%, 91%, 92%, 93%, 94%, 95%, 96%, 97%, 98% or 99% identical to SEQ ID NO: 6; and
b. a second peptide comprising an amino acid sequence of: SEQ ID NO: 3 or an amino acid sequence that is at least 65%, 70%, 75%, 80%, 85%, 90%, 91%, 92%, 93%, 94%, 95%, 96%, 97%, 98% or 99% identical to SEQ ID NO: 3, or a variant of a peptide comprising an amino acid sequence of SEQ ID NO: 3, wherein the amino acid sequence has an amino acid variant on position N29, and which substantially retains the biological activity of the peptide.

An embodiment of the present invention relates to a composition comprising:
a. a cyclic peptide comprising an amino acid sequence of: SEQ ID NO: 6, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 6; and
b. a second peptide comprising an amino acid sequence of: SEQ ID NO: 3, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 3.

An embodiment of the present invention relates to a composition comprising:
a. a cyclic peptide comprising an amino acid sequence of: SEQ ID NO: 6, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 6; and
b. a second peptide comprising an amino acid sequence of: SEQ ID NO: 3, or homologues thereof, or an amino acid sequence that is at least 65%, 70%, 75%, 80%, 85%, 90%, 91%, 92%, 93%, 94%, 95%, 96%, 97%, 98% or 99% identical to SEQ ID NO: 3, or a variant of a peptide comprising an amino acid sequence of SEQ ID NO: 3, wherein the amino acid sequence has an amino acid variant on position N29, and which substantially retains the biological activity of the peptide.

Yet another embodiment of the present invention relates to a composition comprising:
a. at least one cyclic peptide comprising an amino acid sequence of any of SEQ ID NOs: 1, 2 or 6, or an amino acid sequence that is at least 90%, 91%, 92%, 93%, 94%, 95%, 96%, 97%, 98% or 99% identical to any of SEQ ID NO: 1, 2 or 6; and
b. at least one second peptide comprising an amino acid sequence of any of SEQ ID NOs: 3 to 5, or an amino acid sequence that is at least 90%, 91%, 92%, 93%, 94%, 95%, 96%, 97%, 98% or 99% identical to any of SEQ ID NOs: 3 to 5, or a variant of a peptide comprising an amino acid sequence of SEQ ID NO: 3, wherein the amino acid sequence has an amino acid variant on position N29, and which retains the biological activity of the peptide.

Another embodiment of the present invention relates to a composition comprising:
a. at least one cyclic peptide comprising an amino acid sequence of any of SEQ ID NOs: 1, 2 or 6, or an amino acid sequence that is at least 85% identical to any of SEQ ID NO: 1, 2 or 6; and
b. at least one second peptide comprising an amino acid sequence of any of SEQ ID NOs: 3 to 5, or an amino acid sequence that is at least 85% identical to any of SEQ ID NOs: 3 to 5, or a variant of a peptide comprising an amino acid sequence of SEQ ID NO: 3, wherein the amino acid sequence has an amino acid variant on position N29, and which retains the biological activity of the peptide.

Another embodiment of the present invention relates to a composition comprising:
a. at least one cyclic peptide comprising an amino acid sequence of any of SEQ ID NOs: 1, 2 or 6, or an amino acid sequence that is at least 90% identical to any of SEQ ID NO: 1, 2 or 6; and
b. at least one second peptide comprising an amino acid sequence of any of SEQ ID NOs: 3 to 5, or an amino acid sequence that is at least 90% identical to any of SEQ ID NOs: 3 to 5, or a variant of a peptide comprising an amino acid sequence of SEQ ID NO: 3, wherein the amino acid sequence has an amino acid variant on position N29, and which retains the biological activity of the peptide.

Another embodiment of the present invention relates to a composition comprising:
a. at least one cyclic peptide comprising an amino acid sequence of any of SEQ ID NOs: 1, 2 or 6, or an amino acid sequence that is at least 92% identical to any of SEQ ID NO: 1, 2 or 6; and
b. at least one second peptide comprising an amino acid sequence of any of SEQ ID NOs: 3 to 5, or an amino acid sequence that is at least 92% identical to any of SEQ ID NOs: 3 to 5, or a variant of a peptide comprising an amino acid sequence of SEQ ID NO: 3, wherein the amino acid sequence has an amino acid variant on position N29, and which retains the biological activity of the peptide.

Another embodiment of the present invention relates to a composition comprising:
a. at least one cyclic peptide comprising an amino acid sequence of any of SEQ ID NOs: 1, 2 or 6, or an amino acid sequence that is at least 94% identical to any of SEQ ID NO: 1, 2 or 6; and
b. at least one second peptide comprising an amino acid sequence of any of SEQ ID NOs: 3 to 5, or an amino acid sequence that is at least 94% identical to any of SEQ ID NOs: 3 to 5, or a variant of a peptide comprising an amino acid sequence of SEQ ID NO: 3, wherein the amino acid sequence has an amino acid variant on position N29, and which retains the biological activity of the peptide.

Another embodiment of the present invention relates to a composition comprising:
a. at least one cyclic peptide comprising an amino acid sequence of any of SEQ ID NOs: 1, 2 or 6, or an amino acid sequence that is at least 96% identical to any of SEQ ID NO: 1, 2 or 6; and
b. at least one second peptide comprising an amino acid sequence of any of SEQ ID NOs: 3 to 5, or an amino acid sequence that is at least 96% identical to any of SEQ ID NOs: 3 to 5, or a variant of a peptide comprising an amino acid sequence of SEQ ID NO: 3, wherein the amino acid sequence has an amino acid variant on position N29, and which retains the biological activity of the peptide.

Another embodiment of the present invention relates to a composition comprising:
a. at least one cyclic peptide comprising an amino acid sequence of any of SEQ ID NOs: 1, 2 or 6, or an amino acid sequence that is at least 98% identical to any of SEQ ID NO: 1, 2 or 6; and
b. at least one second peptide comprising an amino acid sequence of any of SEQ ID NOs: 3 to 5, or an amino acid sequence that is at least 98% identical to any of SEQ ID NOs: 3 to 5, or a variant of a peptide comprising an amino acid sequence of SEQ ID NO: 3, wherein the amino acid sequence has an amino acid variant on position N29, and which retains the biological activity of the peptide.

Yet another embodiment of the present invention relates to a composition comprising:
a. at least one cyclic peptide comprising an amino acid sequence of any of SEQ ID NOs: 1, 2 or 6, or an amino acid sequence that is at least 90%, 91%, 92%, 93%, 94%, 95%, 96%, 97%, 98% or 99% identical to any of SEQ ID NO: 1, 2 or 6; and
b. at least one second peptide comprising an amino acid sequence of SEQ ID NO: 3, or an amino acid sequence that is at least 90%, 91%, 92%, 93%, 94%, 95%, 96%, 97%, 98% or 99% identical to SEQ ID NO: 3, or a variant of a peptide comprising an amino acid sequence of SEQ ID NO: 3, wherein the amino acid sequence has an amino acid variant on position N29, and which retains the biological activity of the peptide.

Another embodiment of the present invention relates to a composition comprising:
a. at least one cyclic peptide comprising an amino acid sequence of any of SEQ ID NOs: 1, 2 or 6, or an amino acid sequence that is at least 85% identical to any of SEQ ID NO: 1, 2 or 6; and
b. at least one second peptide comprising an amino acid sequence of SEQ ID NO: 3, or an amino acid sequence that is at least 85% identical to SEQ ID NO: 3, or a variant of a peptide comprising an amino acid sequence of SEQ ID NO: 3, wherein the amino acid sequence has an amino acid variant on position N29, and which retains the biological activity of the peptide.

Another embodiment of the present invention relates to a composition comprising:
a. at least one cyclic peptide comprising an amino acid sequence of any of SEQ ID NOs: 1, 2 or 6, or an amino acid sequence that is at least 90% identical to any of SEQ ID NO: 1, 2 or 6; and
b. at least one second peptide comprising an amino acid sequence of SEQ ID NO: 3, or an amino acid sequence that is at least 90% identical to SEQ ID NO: 3, or a variant of a peptide comprising an amino acid sequence of SEQ ID NO: 3, wherein the amino acid sequence has an amino acid variant on position N29, and which retains the biological activity of the peptide.

Another embodiment of the present invention relates to a composition comprising:
a. at least one cyclic peptide comprising an amino acid sequence of any of SEQ ID NOs: 1, 2 or 6, or an amino acid sequence that is at least 92% identical to any of SEQ ID NO: 1, 2 or 6; and
b. at least one second peptide comprising an amino acid sequence of SEQ ID NO: 3, or an amino acid sequence that is at least 92% identical to SEQ ID NO: 3, or a variant of a peptide comprising an amino acid sequence of SEQ ID NO: 3, wherein the amino acid sequence has an amino acid variant on position N29, and which retains the biological activity of the peptide.

Another embodiment of the present invention relates to a composition comprising:
a. at least one cyclic peptide comprising an amino acid sequence of any of SEQ ID NOs: 1, 2 or 6, or an amino acid sequence that is at least 94% identical to any of SEQ ID NO: 1, 2 or 6; and
b. at least one second peptide comprising an amino acid sequence of SEQ ID NO: 3, or an amino acid sequence that is at least 94% identical to SEQ ID NO: 3, or a variant of a peptide comprising an amino acid sequence of SEQ ID NO: 3, wherein the amino acid sequence has an amino acid variant on position N29, and which retains the biological activity of the peptide.

Another embodiment of the present invention relates to a composition comprising:
a. at least one cyclic peptide comprising an amino acid sequence of any of SEQ ID NOs: 1, 2 or 6, or an amino acid sequence that is at least 96% identical to any of SEQ ID NO: 1, 2 or 6; and
b. at least one second peptide comprising an amino acid sequence of SEQ ID NO: 3, or an amino acid sequence that is at least 96% identical to SEQ ID NO: 3, or a variant of a peptide comprising an amino acid sequence of SEQ ID NO: 3, wherein the amino acid sequence has an amino acid variant on position N29, and which retains the biological activity of the peptide.

Another embodiment of the present invention relates to a composition comprising:
a. at least one cyclic peptide comprising an amino acid sequence of any of SEQ ID NOs: 1, 2 or 6, or an amino acid sequence that is at least 98% identical to any of SEQ ID NO: 1, 2 or 6; and
b. at least one second peptide comprising an amino acid sequence of SEQ ID NO: 3, or an amino acid sequence that is at least 98% identical to SEQ ID NO: 3, or a variant of a peptide comprising an amino acid sequence of SEQ ID NO: 3, wherein the amino acid sequence has an amino acid variant on position N29, and which retains the biological activity of the peptide.

Yet another embodiment of the present invention relates to a composition comprising:
a. at least one cyclic peptide comprising an amino acid sequence of any of SEQ ID NOs: 1, 2 or 6, or an amino acid sequence that is at least 90%, 91%, 92%, 93%, 94%, 95%, 96%, 97%, 98% or 99% identical to any of SEQ ID NO: 1, 2 or 6; and
b. at least one second peptide comprising an amino acid sequence of SEQ ID NO: 4, or an amino acid sequence that is at least 90%, 91%, 92%, 93%, 94%, 95%, 96%, 97%, 98% or 99% identical to SEQ ID NO: 4.

Another embodiment of the present invention relates to a composition comprising:
a. at least one cyclic peptide comprising an amino acid sequence of any of SEQ ID NOs: 1, 2 or 6, or an amino acid sequence that is at least 85% identical to any of SEQ ID NO: 1, 2 or 6; and
b. at least one second peptide comprising an amino acid sequence of SEQ ID NO: 4, or an amino acid sequence that is at least 85% identical to SEQ ID NO: 4.

Another embodiment of the present invention relates to a composition comprising:
a. at least one cyclic peptide comprising an amino acid sequence of any of SEQ ID NOs: 1, 2 or 6, or an amino acid sequence that is at least 90% identical to any of SEQ ID NO: 1, 2 or 6; and
b. at least one second peptide comprising an amino acid sequence of SEQ ID NO: 4, or an amino acid sequence that is at least 90% identical to SEQ ID NO: 4.

Another embodiment of the present invention relates to a composition comprising:
a. at least one cyclic peptide comprising an amino acid sequence of any of SEQ ID NOs: 1, 2 or 6, or an amino acid sequence that is at least 92% identical to any of SEQ ID NO: 1, 2 or 6; and
b. at least one second peptide comprising an amino acid sequence of SEQ ID NO: 4, or an amino acid sequence that is at least 92% identical to SEQ ID NO: 4.

Another embodiment of the present invention relates to a composition comprising:
a. at least one cyclic peptide comprising an amino acid sequence of any of SEQ ID NOs: 1, 2 or 6, or an amino acid sequence that is at least 94% identical to any of SEQ ID NO: 1, 2 or 6; and
b. at least one second peptide comprising an amino acid sequence of SEQ ID NO: 4, or an amino acid sequence that is at least 94% identical to SEQ ID NO: 4.

Another embodiment of the present invention relates to a composition comprising:
a. at least one cyclic peptide comprising an amino acid sequence of any of SEQ ID NOs: 1, 2 or 6, or an amino acid sequence that is at least 96% identical to any of SEQ ID NO: 1, 2 or 6; and
b. at least one second peptide comprising an amino acid sequence of SEQ ID NO: 4, or an amino acid sequence that is at least 96% identical to SEQ ID NO: 4.

Another embodiment of the present invention relates to a composition comprising:
a. at least one cyclic peptide comprising an amino acid sequence of any of SEQ ID NOs: 1, 2 or 6, or an amino acid sequence that is at least 98% identical to any of SEQ ID NO: 1, 2 or 6; and
b. at least one second peptide comprising an amino acid sequence of SEQ ID NO: 4, or an amino acid sequence that is at least 98% identical to SEQ ID NO: 4.

Yet another embodiment of the present invention relates to a composition comprising:
a. at least one cyclic peptide comprising an amino acid sequence of any of SEQ ID NOs: 1, 2 or 6, or an amino acid sequence that is at least 90%, 91%, 92%, 93%, 94%, 95%, 96%, 97%, 98% or 99% identical to any of SEQ ID NO: 1, 2 or 6; and
b. at least one second peptide comprising an amino acid sequence of SEQ ID NO: 5, or an amino acid sequence that is at least 90%, 91%, 92%, 93%, 94%, 95%, 96%, 97%, 98% or 99% identical to SEQ ID NO: 5.

Another embodiment of the present invention relates to a composition comprising:
a. at least one cyclic peptide comprising an amino acid sequence of any of SEQ ID NOs: 1, 2 or 6, or an amino acid sequence that is at least 85% identical to any of SEQ ID NO: 1, 2 or 6; and
b. at least one second peptide comprising an amino acid sequence of SEQ ID NO: 5, or an amino acid sequence that is at least 85% identical to SEQ ID NO: 5.

Another embodiment of the present invention relates to a composition comprising:
a. at least one cyclic peptide comprising an amino acid sequence of any of SEQ ID NOs: 1, 2 or 6, or an amino acid sequence that is at least 90% identical to any of SEQ ID NO: 1, 2 or 6; and
b. at least one second peptide comprising an amino acid sequence of SEQ ID NO: 5, or an amino acid sequence that is at least 90% identical to SEQ ID NO: 5.

Another embodiment of the present invention relates to a composition comprising:
a. at least one cyclic peptide comprising an amino acid sequence of any of SEQ ID NOs: 1, 2 or 6, or an amino acid sequence that is at least 92% identical to any of SEQ ID NO: 1, 2 or 6; and
b. at least one second peptide comprising an amino acid sequence of SEQ ID NO: 5, or an amino acid sequence that is at least 92% identical to SEQ ID NO: 5.

Another embodiment of the present invention relates to a composition comprising:
a. at least one cyclic peptide comprising an amino acid sequence of any of SEQ ID NOs: 1, 2 or 6, or an amino acid sequence that is at least 94% identical to any of SEQ ID NO: 1, 2 or 6; and
b. at least one second peptide comprising an amino acid sequence of SEQ ID NO: 5, or an amino acid sequence that is at least 94% identical to SEQ ID NO: 5.

Another embodiment of the present invention relates to a composition comprising:
a. at least one cyclic peptide comprising an amino acid sequence of any of SEQ ID NOs: 1, 2 or 6, or an amino acid sequence that is at least 96% identical to any of SEQ ID NO: 1, 2 or 6; and
b. at least one second peptide comprising an amino acid sequence of SEQ ID NO: 5, or an amino acid sequence that is at least 96% identical to SEQ ID NO: 5.

Another embodiment of the present invention relates to a composition comprising:
a. at least one cyclic peptide comprising an amino acid sequence of any of SEQ ID NOs: 1, 2 or 6, or an amino acid sequence that is at least 98% identical to any of SEQ ID NO: 1, 2 or 6; and
b. at least one second peptide comprising an amino acid sequence of SEQ ID NO: 5, or an amino acid sequence that is at least 98% identical to SEQ ID NO: 5.

The following embodiments of the present invention relate to a composition comprising a cyclic peptide comprising an amino acid sequence of a SEQ ID NO of one cyclic peptide as listed in Table 1 and a second peptide comprising an amino acid sequence of a SEQ ID NO of one second peptide as listed in Table 1. One such embodiment is a composition comprising a cyclic peptide comprising an amino acid sequence of SEQ ID NO: 2 and a second peptide comprising an amino acid sequence of SEQ ID NO: 4. Another such an embodiment is a composition comprising a cyclic peptide comprising an amino acid sequence of SEQ ID NO: 1 and a second peptide comprising an amino acid sequence of SEQ ID NO: 5.

TABLE 1

| cyclic peptide | second peptide |
|---|---|
| SEQ ID NO: | SEQ ID NO: |
| 1: GLPVCGETCVGGTCNTPG | 3: SPTCIPSGQPCPYNENCC |
| CTCSWPVCTRN | SQSCTFKENENGNTVKRCD |
| 2: GLPVCGETCFGGTCNTPG | 4: RSCCPCYWGGCPWGQNCY |
| CSCTWPICTRD | PEGCSGPKV |
| 6: GLPVCGETCVGGTCNTPG | 5: GSQYCVPVDQPCSLNTQP |
| CTCSWPKCTRN | CCDDATCTQERNENGHTVYYC |
| | RA |

SEQ ID NO: 33, which has amino acid sequence SPTCIPSGQPCPYNENCCSQSCTFKENETGNTVKRCD, and which is also known as ω-hexatoxin-Ar1f, is a native peptide of Atrax robustus.

At least one described herein is understood to mean one or more, preferably 1, 2 or 3, preferably 1 or 2, preferably 1.

Variants, functional variants and homologues of the sequences described herein are also encompassed by the embodiments of the present invention. Variants herein are understood to refer to peptides having substantially similar amino acid sequences. Functional variants herein are understood to refer to variants that are biologically active, herein understood to mean that they retain the biological activity of the native peptide sequence, herein understood to mean insecticidal activity as described herein. Homologues herein are understood to mean peptides having sequence similarity to sequences according to the present application, which are suitable for the processes described herein, such as insecticidal activity, and which are naturally occurring sequences.

Variants, functional variants and homologues as described herein encompass peptides that are derived from native peptides by substitution, insertion, deletion or addition of at least one amino acid on at least one position in the native peptide. Substitution, insertion or addition of at least one amino acid on at least one position in the native peptide comprises the substitution, insertion or addition by a non-proteinogenic amino acid. Non-proteinogenic amino acids herein are understood to mean amino acids that are not naturally encoded or found in the genetic code of any organism and are not translationally incorporated into proteins. They comprise any organic compound with an amine and a carboxylic acid functional group. Non-limitative examples of these are phenylalanine derivatives such as 4-methyl-phenylalanine and 3,4-dihydroxy-phenylalanine; phenylglycine derivatives such as 4-hydroxy-phenylglycine; tryptophan derivatives such as 6-amino-7-hydroxy-l-tryptophan; methionine derivatives such as nitrilo-l-methionine; alanine derivatives such as adamanthane; cysteine derivatives such as penicillamine; asparagine/glutamine derivatives such as cysteine-s-acetamide; lysine derivatives such as 2,3-diaminopropanoic acid; arginine derivatives such as c-gamma-hydroxy arginine; serine/threonine derivatives such as homoserine and phosphothreonine; histidine derivatives such as 2-fluoro-l-histidine and asparagine/glutamine derivatives such as I-2-amino-6-methylene-pimelic acid and 4-fluoro-glutamic acid. More examples are for example illustrated in the SwissSidechain database (http://www.swisssidechain.ch) (Gfeller D. et al., 2013).

Substantially similar amino acid sequences have only a small number of sequence changes, for example in non-conserved residues.

Functional variants also comprise variants that have sequence changes that do not affect function, for example in non-conserved residues.

Changes in a nucleic acid sequence by mutation, substitution, insertion, deletion or addition that lead to changes in the amino acid sequence of the encoded polypeptide, but without altering its biological activity (i.e. having insecticidal activity), are well-known in the art. These are also known as conservative alterations. For example, a codon for alanine, a hydrophobic amino acid, may be substituted by a codon encoding another less hydrophobic amino acid, such as glycine, or a more hydrophobic amino acid, such as valine, leucine, or isoleucine. Similarly, changes which result in substitution of one negatively charged amino acid for another, such as aspartic acid for glutamic acid, or one positively charged amino acid for another, such as lysine for arginine, can also be expected to produce a functionally equivalent product. Nucleotide changes at the N-terminal and/or C-terminal ends of a non-cyclic peptide are also expected to not alter the biological activity of the non-cyclic peptide. Methods for introducing nucleotide changes are well-known in the art. The changes in the nucleic acid sequence are not expected to generate profound changes with regard to the biological activity. When such changes are difficult to predict the biological activity of the peptide, routine screening methods such as insect-feeding assays can be employed.

Methods for introducing nucleotide changes in a sequence, for example a sequence encoding SEQ ID NO: 3, thereby preferably altering the encoded amino acid, are well-known in the art and also referred to as methods for mutagenesis. Examples of methods for mutagenesis are cassette mutagenesis, Kunkel's method, PCR site-directed mutagenesis, site saturation mutagenesis, seamless ligation cloning extract, artificial gene synthesis and CRISPR-Cas9.

It was found that variants of peptides comprising an amino acid sequence of SEQ ID NO: 3, wherein the amino acid sequence has an amino acid variant on position N29, substantially retain the biological activity of the peptide, while also having an increased stability at alkaline pH compared to the non-variant peptide, wherein the increased stability is in an alkaline medium of greater than pH 7.

Accordingly, a variant of a peptide comprising an amino acid sequence of SEQ ID NO: 3, wherein the amino acid sequence has an amino acid variant on position N29, and which substantially retains the biological activity of the peptide, can be an amino acid variant of A, R, D, C, Q, E, G, H, I, L, K, M, F, P, S, T, W, Y or V.

A variant of a peptide comprising an amino acid sequence of SEQ ID NO: 3, wherein the amino acid sequence has an amino acid variant on position N29, and which substantially retains the biological activity of the peptide, can be an amino acid variant of A, R, D, C, Q, E, G, H, I, L, K, M, F, P, S, T, W, Y or V; with the proviso that the peptide does not comprise the amino acid sequence of: SEQ ID NO: 33.

A variant of a peptide comprising an amino acid sequence of SEQ ID NO: 3, wherein the amino acid sequence has an amino acid variant on position N29, and which substantially retains the biological activity of the peptide, can be an amino acid variant of A, R, C, Q, E, G, H, I, L, K, M, F, P, S, T, W, Y or V.

A variant of a peptide comprising an amino acid sequence of SEQ ID NO: 3, wherein the amino acid sequence has an amino acid variant on position N29, and which substantially retains the biological activity of the peptide, can be an amino acid variant of A, R, C, Q, E, G, H, I, L, K, M, F, P, S, T, W, Y or V; with the proviso that the peptide does not comprise the amino acid sequence of: SEQ ID NO: 33.

A variant of a peptide comprising an amino acid sequence of SEQ ID NO: 3, wherein the amino acid sequence has an amino acid variant on position N29, and which substantially retains the biological activity of the peptide, can be an amino acid variant of A, R, C, G, H, I, L, K, M, F, P, S, T, W, Y or V.

A variant of a peptide comprising an amino acid sequence of SEQ ID NO: 3, wherein the amino acid sequence has an amino acid variant on position N29, and which substantially retains the biological activity of the peptide, can be an amino acid variant of A, R, C, G, H, I, L, K, M, F, P, S, T, W, Y or V; with the proviso that the peptide does not comprise the amino acid sequence of: SEQ ID NO: 33.

A variant of a peptide comprising an amino acid sequence of SEQ ID NO: 3, wherein the amino acid sequence has an amino acid variant on position N29, and which substantially retains the biological activity of the peptide, can be an amino acid variant of A, R, D, C, Q, E, G, H, I, L, K, M, F, P, S, W, Y or V.

A variant of a peptide comprising an amino acid sequence of SEQ ID NO: 3, wherein the amino acid sequence has an amino acid variant on position N29, and which substantially retains the biological activity of the peptide, can be an amino acid variant of A, R, D, C, Q, E, G, H, I, L, K, M, F, P, W, Y or V.

A variant of a peptide comprising an amino acid sequence of SEQ ID NO: 3, wherein the amino acid sequence has an amino acid variant on position N29, and which substantially retains the biological activity of the peptide, can be an amino acid variant of A, C, Q, G, I, L, M, F, P, S, T, W, Y or V.

A variant of a peptide comprising an amino acid sequence of SEQ ID NO: 3, wherein the amino acid sequence has an amino acid variant on position N29, and which substantially retains the biological activity of the peptide, can be an amino acid variant of A, C, Q, G, I, L, M, F, P, S, T, W, Y or V; with the proviso that the peptide does not comprise the amino acid sequence of: SEQ ID NO: 33.

A variant of a peptide comprising an amino acid sequence of SEQ ID NO: 3, wherein the amino acid sequence has an amino acid variant on position N29, and which substantially retains the biological activity of the peptide, can be an amino acid variant of A, C, G, I, L, M, F, P, S, T, W, Y or V.

A variant of a peptide comprising an amino acid sequence of SEQ ID NO: 3, wherein the amino acid sequence has an amino acid variant on position N29, and which substantially retains the biological activity of the peptide, can be an amino acid variant of A, C, G, I, L, M, F, P, S, T, W, Y or V; with the proviso that the peptide does not comprise the amino acid sequence of: SEQ ID NO: 33.

A variant of a peptide comprising an amino acid sequence of SEQ ID NO: 3, wherein the amino acid sequence has an amino acid variant on position N29, and which substantially retains the biological activity of the peptide, can be an amino acid variant of A, C, G, I, L, M, F, P, W or V.

A variant of a peptide comprising an amino acid sequence of SEQ ID NO: 3, wherein the amino acid sequence has an amino acid variant on position N29, and which substantially retains the biological activity of the peptide, can be an amino acid variant of A, L, P or T.

A variant of a peptide comprising an amino acid sequence of SEQ ID NO: 3, wherein the amino acid sequence has an amino acid variant on position N29, and which substantially retains the biological activity of the peptide, can be an amino acid variant of A, L, P or T; with the proviso that the peptide does not comprise the amino acid sequence of: SEQ ID NO: 33.

A variant of a peptide comprising an amino acid sequence of SEQ ID NO: 3, wherein the amino acid sequence has an amino acid variant on position N29, and which substantially retains the biological activity of the peptide, can be an amino acid variant of A, L or P.

A variant of a peptide comprising an amino acid sequence of SEQ ID NO: 3, wherein the amino acid sequence has an amino acid variant on position N29, and which substantially retains the biological activity of the peptide, can be an amino acid variant of A or L.

A variant of a peptide comprising an amino acid sequence of SEQ ID NO: 3, wherein the amino acid sequence has an amino acid variant on position N29, and which substantially retains the biological activity of the peptide, can be an amino acid variant of a non-proteinogenic amino acid.

Preferably, a variant of an amino acid sequence is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to the non-variant amino acid sequence.

Preferably, a functional variant of an amino acid sequence is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to the non-variant amino acid sequence. More preferably, a functional variant is at least 85%, 86%, 87%, 88%, 89%, 90%, 91%, 92%, 93%, 94%, 95%, 96%, 97%, 98% or 99% identical to the non-variant amino acid sequence. Most preferably, a functional variant is at least 90%, 91%, 92%, 93%, 94%, 95%, 96%, 97%, 98% or 99% identical to the non-variant amino acid sequence.

Preferably, a homologue of an amino acid sequence is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to any amino acid sequence according to the invention. More preferably, a homologue of an amino acid sequence is at least 85%, 86%, 87%, 88%, 89%, 90%, 91%, 92%, 93%, 94%, 95%, 96%, 97%, 98% or 99% identical to any amino acid sequence according to the invention.

A percentage identity between any two nucleic acid sequences, amino acid sequences or peptides can be determined via sequence comparison or sequence alignment. Methods of sequence comparison and sequence alignment are well-known in the art and can be determined via manual alignment and visual inspection or an algorithm, which is suitably implemented on a computer. When performing sequence comparison or alignment one sequence is typically used as a reference sequence to which the other sequence is compared. The comparison occurs in a comparison window which is an individually specified contiguous sequence of each of the compared sequences. Additions or deletions relative from one sequence to the other may be included in any of the sequences, thereby introducing so-called gaps in the other sequence. The introduction of gaps can result in a better alignment between the two sequences. However, the amount of gaps in an alignment should be kept to a minimum in order to create a useful alignment, because too many gaps can cause an alignment to become meaningless. To avoid a high sequence identity between two sequences because of the introduction of too many gaps it is known to a person skilled in the art to use a gap penalty in order to compensate. Gap penalties are used to adjust alignment scores based on the number and length of gaps. Examples of gap penalties are constant, linear, affine, convex, and profile-based gap penalties.

Sequence identity or identity with regard to nucleic acid or amino acid sequences or peptides are understood to mean that the two nucleic acid or amino acid sequences or peptides are the same when aligned for maximum correspondence over a comparison window, as measured using sequence comparison or sequence alignment. Identical, percent identical or percent identity in the context of two or more nucleic acid or amino acid sequences or peptides, refer to two or more nucleic acid or amino acid sequences or peptides that are the same or have a specified percentage of amino acid residues or nucleotides that are the same, when compared and aligned for maximum correspondence over a comparison window, as measured using sequence comparison or sequence alignment. A percentage identity with regard to amino acid sequences or peptides where aligned amino acid positions are not identical is often based on conservative alterations, in particular substitutions, as described above, and is expected to produce a functionally equivalent peptide. The percentage identity can be adjusted upwards to correct for conservative alterations and methods for such adjusting are well-known in the art. For clarity, the percentage identity of a sequence; the “target sequence”, for example a sequence listed herein as a SEQ ID NO, is compared and aligned for maximum correspondence over a comparison window, as measured using sequence comparison or sequence alignment, wherein the length of the individually specified contiguous sequence of the comparison window of the target sequence, for example a sequence listed herein as a SEQ ID NO, is the same or substantially the same as the total length of the target sequence.

The cyclic peptide and the second peptide can be used in any ratio in the composition according to the invention. Certain ratios are more preferred. A ratio of at least one cyclic peptide to at least one second peptide of from 50:1 to 1:50 is herein understood to mean that the total amount of molecules of the at least one cyclic peptide is from 50 fold to 1/50^{th} of the at least one second peptide. Accordingly, the composition according to the invention has a ratio of at least one cyclic peptide to at least one second peptide of from 100:1 to 1:100, from 100:1 to 1:50, from 100:1 to 1:10, from 100:1 to 1:1, from 50:1 to 1:100, from 50:1 to 1:50, from 50:1 to 1:10, from 50:1 to 1:1, from 100:1 to 10:1, from 50:1 to 10:1, from 10:1 to 1:100, from 10:1 to 1:50, from 10:1 to 1:10, from 10:1 to 1:1, from 5:1 to 1:1, from 5:1 to 1:5, from 1:1 to 1:100, from 1:1 to 1:50, from 1:1 to 1:10, from 1:1 to 1:5, from 1:10 to 1:50, from 1:10 to 1:100 or from 1:50 to 1:100. Preferably, the ratio is from 10:1 to 1:100, from 10:1 to 1:50, from 10:1 to 1:10, from 1:1 to 1:100, from 1:1 to 1:50, from 5:1 to 1:5 or from 1:1 to 1:10.

Another aspect of the invention relates to the cyclic peptide or second peptide having insecticidal activity when ingested by an insect. Preferably, the cyclic peptide has insecticidal activity when ingested by an insect. Preferably, the second peptide has insecticidal activity when ingested by an insect.

To ingest is herein understood to mean to take into the body via the mouth. Insects possess a complete digestive system consisting of the alimentary canal, an enclosed tube which runs lengthwise through the body from mouth to anus. Food enters the mouth and is processed as it progresses to the anus.

It is common practice in the art to present insecticidal compounds on or near the plant, plant tissue or plant propagation material in order to have the most effective distribution of the insecticidal compounds towards the insect. Hereby, insecticidal compounds are delivered to the insect so that they can exert their insecticidal effects.

Accordingly, another aspect of the invention relates to a plant, plant tissue or plant propagation material comprising a composition according to the present invention, or having a composition according to the present invention adhered thereto. Preferably, the invention relates to a plant, plant tissue or plant propagation material comprising a composition according to the present invention. Preferably, the invention relates to a plant, plant tissue or plant propagation material having a composition according to the present invention adhered thereto.

Another aspect of the invention relates to a plant, plant tissue or plant propagation material comprising a composition according to the present invention, or having a composition according to the present invention applied thereto. Preferably, the invention relates to a plant, plant tissue or plant propagation material comprising a composition according to the present invention. Preferably, the invention relates to a plant, plant tissue or plant propagation material having a composition according to the present invention applied thereto.

Plant is herein understood to mean all physical parts of a plant, including seeds, seedlings, saplings, roots, tubers, stems, stalks, foliage and fruits.

Plant tissue is herein understood to mean any type of tissue of a plant, such as epidermis, vascular tissue, ground tissue, xylem, phloem, parenchyma, collenchyma or sclerenchyma.

Plant propagation material is herein understood to mean plant material from which a new plant can be grown, in particular seeds. This also includes plant material suitable for vegetative reproduction and plant cuttings, roots, fruits, tubers, bulbs, corms and rhizomes.

A plant, plant tissue, plant cell or plant propagation material comprising a composition according to the present invention comprises a plant, plant tissue, plant cell or plant propagation material expressing at least one peptide of the composition. Preferably, the plant, plant tissue, plant cell or plant propagation material expresses at least one cyclic peptide of the composition. Preferably, the plant, plant tissue, plant cell or plant propagation material expresses at least one second peptide of the composition. More preferably, the plant, plant tissue, plant cell or plant propagation material expresses at least one cyclic peptide and at least one second peptide of the composition. By expressing one or both components of the composition in the plant, plant tissue, plant cell or plant propagation material the insect ingests the insecticidal peptide while feeding on the plant, plant tissue, plant cell or plant propagation material, thereby controlling and preferably killing the insect.

To express is herein understood to mean transient or stable expression of the at least one cyclic peptide and/or at least one second peptide of the composition and/or a polynucleotide sequence that encodes for the at least one cyclic peptide and/or at least one second peptide of the composition. Stable expression comprises integration of the polynucleotide sequence into the genome of the target organism. Enabling transient or stable expression in a plant, plant tissue, plant cell, plant propagation material or another organism, thereby generating a transformed, transgenic or genetically modified organism is achievable by routine procedures and well-known methods in the art.

In addition to expressing the at least one cyclic peptide and/or at least one second peptide of the composition in a plant each of the components of the composition can also be expressed in other organisms such as viruses, fungi, protozoa, bacteria and nematodes, thereby providing another vector of delivery of the peptides of the composition according to the invention to the insect.

An aspect of the current invention is a method for delivering a composition to an insect, the method comprising applying the composition according to the invention to the plant, plant locus, plant tissue or plant propagation material, to obtain a treated material.

Another aspect of the invention is a method for delivering a composition to an insect, the method comprising applying the composition according to the invention to the plant, plant locus, plant tissue or plant propagation material, to obtain a treated material, wherein the insecticidal composition comprises a cyclic peptide and/or a second peptide.

Plant locus is herein understood to mean the locus or the location where the plant or plant propagation material is placed or to be placed. The plant locus includes soil and other vegetation.

Specific methods for applying or administering a composition according to the invention are known in the art. Examples of these are spraying, dusting, foliar spraying, brushing, spreading, rolling, applying to or coating seeds.

The propagation material can be treated with the composition during or prior to use such as sowing. The composition can also be applied to seed kernels, either by soaking the kernels in a liquid composition or by coating them with a solid composition.

The composition according to the invention can also be delivered to insects by other means such as by injection, thereby exerting its insecticidal effects directly at the desired site of action of the insecticidal peptide.

The active ingredients of the compositions of the present invention are normally applied in compositions further comprising other compounds. The compositions according to the invention can further comprise agriculturally acceptable solid or liquid auxiliaries known in the art such as carriers, surfactants, stabilizers, antifoams, preservatives, binders, solvents, dispersants or fertilizers.

They can also comprise other herbicides or other pesticides such as chemical insecticides, fungicides or nematicides. The compositions can also be microencapsulated, and such compositions can be prepared using suitable techniques known in the art.

Examples of application formulations of compositions according to the invention that can be used for the method according to the invention are solutions, granules, dusts, sprayable powders, emulsion concentrates, coated granules and suspension concentrates. The formulations can be prepared as pest-ingestible formulations. The suitability such formulations can be enhanced by adding ethanol or mono- or disaccharide compounds such as glucose, sucrose, fructose or lactose.

Typical rates of concentration of active ingredient are of from 180 g to 12.0 kg of active ingredient a.i.) per hectare (ha), preferably of from 10 g to 1.0 kg a.i./ha. most preferably of from 20 g to 600 g a.i./ha. Active ingredient is understood to mean either the cyclic peptide or the second peptide according to the composition of the invention.

As described before the purpose of an insecticide is to provide a material that reduces damage caused by insect pests. Accordingly, an aspect of the current invention is to provide a method for controlling insects, comprising applying a composition according to the invention to an insect or its environment, a plant, plant tissue or plant propagation material or the locus where the plant or plant propagation material is planted.

The term “insecticidal” herein is understood to mean to kill, to destroy, to retard or prevent growth, movement or feeding or to inhibit reproduction of other animal pests than strictly insects, including acarina and nematodes, or other pests susceptible to the treatment by a chemical compound or composition comprising two or more active ingredients, e.g. one or more or peptides. Accordingly, the term “insecticidal” herein is understood to encompass insecticidal, acaricidal and/or nematicidal activity.

To control is herein understood to mean to kill, to destroy, to retard or prevent growth, movement or feeding, to reduce physical fitness or to inhibit reproduction. In addition, an aspect of the current invention is to provide a use of a composition according to the invention for controlling insects or otherwise animal pests.

The compositions described herein can be used in a method for controlling insects with the exception of a method for treatment of the human or animal body by surgery or therapy and diagnostic methods practiced on the human or animal body.

The compositions according to the invention can be used to control insect pests and/or other pests, including pests from the aracidae and nematode, trematodes and/or mollusc family.

Preferred insects are insects of the order Lepidoptera such as Spodoptera littoralis, Plutella xylostella, Cydia pomonella.

As used herein, the term “insecticidal” activity refers to the activity of an agent, in particular a combination of synergistic substances, that exhibit a measurable effect on pest, in particular insect fitness, which may comprise, but is not limited to insect mortality, pest, such as insect weight loss, insect repellence, and other behavioural and physical changes of an insect after feeding and/or exposure for an appropriate length of time. In this manner, the insecticidal activity impacts at least one measurable parameter of insect fitness. Accordingly, “insecticidal agent” will act similarly to suppress, control, and/or kill an invading pathogen.

The term “insecticidal composition” is intended to mean that the compositions of embodiments of the invention have activity against plant insect pathogens, and thus are capable of suppressing, controlling, and/or killing the invading insect. An insecticidal composition of the embodiments of the invention will reduce the symptoms resulting from insect challenge by at least about 5% to about 50%, at least about 10% to about 60%, at least about 30% to about 70%, at least about 40% to about 80%, or at least about 50% to about 90% or greater. Hence, the methods of the embodiments of the invention can be utilized to protect organisms, particularly plants, from invading insects.

Assays that measure insecticidal activity are commonly known in the art, such as insect-feeding bioassays. See, for example, Marrone et al. (1985) J. Econ. Entomol. 78:290-293 and Czapla and Lang (1990) J. Econ. Entomol. 83:2480-2485. Methods of rearing insect larvae and performing bioassays are well known to one of ordinary skill in the art. A wide variety of bioassay techniques are known to one skilled in the art.

General procedures include addition of the experimental compound or organism to the diet source in an enclosed container. Insecticidal activity can be measured by, but is not limited to, changes in mortality, weight loss, attraction, repellence and other behavioural and physical changes after feeding and exposure for an appropriate length of time. Bioassays described herein can be used with any feeding insect pest in the larval or adult stage.

As mentioned before, cyclotides in general have been shown to have not only insecticidal activity, but also toxic activity against nematodes, trematodes and mollusks. The mode of action by which kalata B1 and B2 are thought to disrupt cell membranes (by binding to phosphatidylethanolamine and to form lipidic toroidal pores) is a principle that can be applied to cells of any organism. In addition, other insecticidal peptides such as the second insecticidal peptides herein can be toxic to non-insect organisms. For example, Av3 has been shown to be toxic to crustaceans, GS-ω/κ-Hxtx-Hv1h is able to control spider mites and broad mites. Therefore, other pest organisms can also be advantageously controlled by the composition according to the invention. The insecticidal peptides described herein also have an increased pesticidal effect when applied as a composition compared to when applied alone.

As used herein, the terms “pesticidal activity” and “insecticidal activity” are used synonymously to refer to activity of an organism or a substance (such as, for example, a protein) that can be measured by but is not limited to pest mortality, pest weight loss, pest repellency, and other behavioral and physical changes of a pest after feeding and exposure for an appropriate length of time.

Pesticides are herein understood to mean substances that are able to control pests. The composition according to the invention can thus be considered to function as a pesticide. Pesticides comprise insecticides, herbicides, nematicides, molluscicides, fungicides and bactericides. Pesticides can for example be in the form of peptides, proteins, microbial agents or chemical pesticides.

Pests are herein understood to mean organisms that cause harm to other organisms, in particular to agricultural crops. To cause harm comprises to kill, to destroy (parts of), to feed upon, to retard or prevent growth, to reduce physical fitness and to inhibit reproduction.

The composition according to the invention can be used in combination with other compounds, including other pesticides such as insecticides, fungicides, or agents that enhance the activity of the composition according to the invention, in for example chemical treatment or pest control programs. The combination may have further surprising advantages, which could be described as synergistic effects.

Suitable other compounds are, for example, compounds of the following classes of active ingredients: organophosphates, nitrophenol derivatives, thioureas, juvenile hormones, formamidines, benzophenone derivatives, ureas, pyrrole derivatives, carbamates, pyrethroids, chlorinated hydrocarbons, acylureas, pyridylmethyleneamino derivatives, macrolides, benzoylureas, neonicotinoids and biological agents such as Bacillus thurigiensis strains or bacterially-derived pesticides such as spinosads, avermectins and Cry proteins.

The composition according to the invention can be used in any ratio in combination with other compounds, including other pesticides, insecticides, fungicides or agents that enhance the activity of the composition according to the invention. Certain ratios are more preferred. A ratio of the composition according to the invention to other compounds of from 50:1 to 1:50 is herein understood to mean that the total amount of molecules of the composition according to the invention is from 50 fold to 1/50^{th} of the other compound, on a weight basis. Accordingly, the composition according to the invention has a ratio of at least one cyclic peptide to at least one second peptide of from 100:1 to 1:100, from 100:1 to 1:50, from 100:1 to 1:10, from 100:1 to 1:1, from 50:1 to 1:100, from 50:1 to 1:50, from 50:1 to 1:10, from 50:1 to 1:1, from 100:1 to 10:1, from 50:1 to 10:1, from 10:1 to 1:100, from 10:1 to 1:50, from 10:1 to 1:10, from 10:1 to 1:1, from 5:1 to 1:1, from 5:1 to 1:5, from 1:1 to 1:100, from 1:1 to 1:50, from 1:1 to 1:10, from 1:1 to 1:5, from 1:10 to 1:50, from 1:10 to 1:100 or from 1:50 to 1:100. Preferably, the ratio is from 10:1 to 1:100, from 10:1 to 1:50, from 10:1 to 1:10, from 1:1 to 1:100, from 1:1 to 1:50, from 5:1 to 1:5 or from 1:1 to 1:10.

The concentrations of the different active ingredients of the combination of the composition according to the invention in combination with other compounds can vary depending on the specific formulation, environmental conditions, methods of application and the extent of pesticidal activity of the individual components.

### Sequences

The present invention provides the specific peptides comprising the amino acid sequences of SEQ ID NOs: 1 to 6 as listed in Table 2.

TABLE 2

| SEQ | | |
|---|---|---|
| ID | | |
| NO: | Peptide name | Amino acid sequence |
| 1 | Kalata B1 (kB1) | GLPVCGETCVGGTCNTPGCTCSWPVCTRN |
| 2 | Kalata B2 (kB2) | GLPVCGETCFGGTCNTPGCSCTWPICTRD |
| 3 | ω-hexatoxin- | SPTCIPSGQPCPYNENCCSQSCTFKENEN |
| | Hv1a | GNTVKRCD |
| 4 | Av3 | RSCCPCYWGGCPWGQNCYPEGCSGPKV |
| 5 | GS-ω/κ- | GSQYCVPVDQPCSLNTQPCCDDATCTQER |
| | hexatoxin-Hv1h | NENGHTVYYCRA |
| 6 | Mutated kB1 | GLPVCGETCVGGTCNTPGCTCSWPKCTRN |
| | (kB1[V25K]) | |

In a further aspect, a skilled person can practice the present invention by applying either simultaneously or in succession, in any order, at least one cyclic peptide of the first aspect with the at least one second peptide of the first aspect to an insect or its environment, a plant, plant tissue or plant propagation material or the locus where the plant or plant propagation material is planted. Accordingly, also made available are:
method for controlling insects, comprising applying (i) at least one cyclic peptide as defined in the first aspect or any one of the embodiment thereof; and (ii) at least one second peptide as defined in the first aspect or any one of the embodiment thereof, either simultaneously or in succession in any order, to an insect or its environment, a plant, plant tissue or plant propagation material or the locus where the plant or plant propagation material is planted;
a method for delivering a composition to an insect, the method comprising applying (i) at least one cyclic peptide as defined in the first aspect or any one of the embodiment thereof; and (ii) at least one second peptide as defined in the first aspect or any one of the embodiment thereof, either simultaneously or in succession in any order, to a plant, plant locus, plant seed or otherwise plant propagation material, to obtain a treated material; and
a method for delivering a composition to an insect, the method comprising applying the composition according to the invention.

The compositions according to the first aspect and the individual peptides when used as a combination (either separately or simultaneously) may be used alone or, preferably, together with the adjuvants conventionally employed in the art of formulation. To this end, it may be conveniently formulated in known manner to emulsifiable concentrates, coatable pastes, directly sprayable ordilutable solutions or suspensions, dilute emulsions, wettable powders, soluble powders, dusts, granulates, and also encapsulations e.g. in polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects.

Suitable carriers and adjuvants, e.g., for agricultural use, can be solid or liquid and are substances useful in formulation technology, e.g. natural or regenerated mineral substances, solvents, dispersants, wetting agents, tackifiers, thickeners, binders or fertilizers. Such carriers are for example described in WO 97/33890.

The compositions, or the individual peptides if applied separately or simultaneously, according to the invention can be applied to the crop area or plant to be treated, simultaneously or in succession with further compounds. These further compounds can be, e.g., fertilizers or micronutrient donors or other preparations, which influence the growth of plants. They can also be selective herbicides or non-selective herbicides as well as insecticides, fungicides, bactericides, nematicides, molluscicides or mixtures of several of these preparations, if desired together with further carriers, surfactants or application promoting adjuvants customarily employed in the art of formulation.

The compositions, or the individual peptides if applied separately or simultaneously, according to the invention have advantageous rates of application that are normally from 180 g to 12.0 kg of active ingredient (a.i.) per hectare (ha), preferably from 10 g to 1.0 kg a.i./ha, most preferably from 20 g to 600 g a.i./ha. Rates for soil application (or drenching) and seed treatment application will differ and the skilled person would be able to determine the suitable rates based on the conditions, soil, crop, etc.

The compositions of the invention, or the individual peptides if applied separately or simultaneously, may be employed in any conventional form, for example in the form of a twin pack, a powder for dry seed treatment (DS), an emulsion for seed treatment (ES), a flowable concentrate for seed treatment (FS), a solution for seed treatment (LS), a water dispersible powder for seed treatment (WS), a capsule suspension for seed treatment (CF), a gel for seed treatment (GF), an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP) or any technically feasible formulation in combination with agriculturally acceptable adjuvants.

Such formulations may be produced in conventional manner, e.g., by mixing the active ingredients with appropriate formulation inerts (diluents, solvents, fillers and optionally other formulating ingredients such as surfactants, biocides, anti-freeze, stickers, thickeners and compounds that provide adjuvancy effects). Also conventional slow release formulations may be employed where long lasting efficacy is intended. Particularly formulations to be applied in spraying forms, such as water dispersible concentrates (e.g. EC, SC, DC, OD, SE, EW, EO and the like), wettable powders and granules, may contain surfactants such as wetting and dispersing agents and other compounds that provide adjuvancy effects, e.g. the condensation product of formaldehyde with naphthalene sulphonate, an alkylarylsulphonate, a lignin sulphonate, a fatty alkyl sulphate, and ethoxylated alkylphenol and an ethoxylated fatty alcohol.

In general, the formulations include from 0.01 to 90% by weight of active agent, from 0 to 20% agriculturally acceptable surfactant and 10 to 99.99% solid or liquid formulation inerts and adjuvant(s), the active agent consisting of at least the compositions according to the first aspect and optionally other active agents, particularly microbiocides or conservatives or the like. Concentrated forms of compositions generally contain in between about 2 and 80%, preferably between about 5 and 70% by weight of active agent. Application forms of formulation may for example contain from 0.01 to 20% by weight, preferably from 0.01 to 5% by weight of active agent. Whereas commercial products will preferably be formulated as concentrates, the end user will normally employ diluted formulations.

The compositions, or the individual peptides if applied separately or simultaneously, according to the invention are preventively and/or curatively valuable active ingredients in the field of pest control, even at low rates of application, which have a very favourable biocidal spectrum and are well tolerated by warm-blooded species, fish and plants. The compositions, or the individual peptides if applied separately or simultaneously, according to the invention act against all or individual developmental stages of normally sensitive, but also resistant, animal pests, such as insects or representatives of the order Acarina. The insecticidal or acaricidal activity of the compositions according to the invention can manifest itself directly, i.e. in destruction of the pests, which takes place either immediately or only after some time has elapsed, for example during ecdysis, or indirectly, for example in a reduced oviposition and/or hatching rate.

Examples of the above-mentioned pests are:
From the order Acarina, for example, Acalitus spp, Aculus spp, Acaricalus spp, Aceria spp, Acarus siro, Amblyomma spp., Argas spp., Boophilus spp., Brevipalpus spp., Bryobia spp, Calipitrimerus spp., Chorioptes spp., Dermanyssus gallinae, Dermatophagoides spp, Eotetranychus spp, Eriophyes spp., Hemitarsonemus spp, Hyalomma spp., Ixodes spp., Olygonychus spp, Ornithodoros spp., Polyphagotarsone latus, Panonychus spp., Phyllocoptruta oleivora, Phytonemus spp, Polyphagotarsonemus spp, Psoroptes spp., Rhipicephalus spp., Rhizoglyphus spp., Sarcoptes spp., Steneotarsonemus spp, Tarsonemus spp. and Tetranychus spp.;
from the order Anoplura, for example,
Haematopinus spp., Linognathus spp., Pediculus spp., Pemphigus spp. and Phylloxera spp.;
from the order Coleoptera, for example, Agriotes spp., Amphimallon majale, Anomala orientalis, Anthonomus spp., Aphodius spp, Astylus atromaculatus, Ataenius spp, Atomaria linearis, Chaetocnema tibialis, Cerotoma spp, Conoderus spp, Cosmopolites spp., Cotinis nitida, Curculio spp., Cyclocephala spp, Dermestes spp., Diabrotica spp., Diloboderus abderus, Epilachna spp., Eremnus spp., Heteronychus arator, Hypothenemus hampei, Lagria vilosa, Leptinotarsa decemlineata, Lissorhoptrus spp., Liogenys spp, Maecolaspis spp, Maladera castanea, Megascelis spp, Melighetes aeneus, Melolontha spp., Myochrous armatus, Orycaephilus spp., Otiorhynchus spp., Phyllophaga spp, Phlyctinus spp., Popillia spp., Psylliodes spp., Rhyssomatus aubtilis, Rhizopertha spp., Scarabeidae, Sitophilus spp., Sitotroga spp., Somaticus spp, Sphenophorus spp, Sternechus subsignatus, Tenebrio spp., Tribolium spp. and Trogoderma spp.;
from the order Diptera, for example, Aedes spp., Anopheles spp, Antherigona soccata, Bactrocea oleae, Bibio hortulanus, Bradysia spp, Calliphora erythrocephala, Ceratitis spp., Chrysomyia spp., Culex spp., Cuterebra spp., Dacus spp., Delia spp, Drosophila melanogaster, Fannia spp., Gastrophilus spp., Geomyza tripunctata, Glossina spp., Hypoderma spp., Hyppobosca spp., Liriomyza spp., Lucilia spp., Melanagromyza spp., Musca spp., Oestrus spp., Orseolia spp., Oscinella frit, Pegomyia hyoscyami, Phorbia spp., Rhagoletis spp, Rivelia quadrifasciata, Scatella spp, Sciara spp., Stomoxys spp., Tabanus spp., Tannia spp. and Tipula spp.;
from the order Hemiptera, for example, Acanthocoris scabrator, Acrosternum spp, Adelphocoris lineolatus, Aleurodes spp., Amblypelta nitida, Bathycoelia thalassina, Blissus spp, Cimex spp., Clavigralla tomentosicollis, Creontiades spp, Distantiella theobroma, Dichelops furcatus, Dysdercus spp., Edessa spp, Euchistus spp., Eurydema pulchrum, Eurygaster spp., Halyomorpha halys, Horcias nobilellus, Leptocorisa spp., Lygus spp, Margarodes spp, Murgantia histrionic, Neomegalotomus spp, Nesidiocoris tenuis, Nezara spp., Nysius simulans, Oebalus insularis, Piesma spp., Piezodorus spp, Rhodnius spp., Sahlbergella singularis, Scaptocoris castanea, Scotinophara spp., Thyanta spp, Triatoma spp., Vatiga illudens; Acyrthosium pisum, Adalges spp, Agalliana ensigera, Agonoscena targionii, Aleurodicus spp, Aleurocanthus spp, Aleurolobus barodensis, Aleurothrixus floccosus, Aleyrodes brassicae, Amarasca biguttula, Amritodus atkinsoni, Aonidiella spp., Aphididae, Aphis spp., Aspidiotus spp., Aulacorthum solani, Bactericera cockerelli, Bemisia spp, Brachycaudus spp, Brevicoryne brassicae, Cacopsylla spp, Cavariella aegopodii Scop., Ceroplaster spp., Chrysomphalus aonidium, Chrysomphalus dictyospermi, Cicadella spp, Cofana spectra, Cryptomyzus spp, Cicadulina spp, Coccus hesperidum, Dalbulus maidis, Dialeurodes spp, Diaphorina citri, Diuraphis noxia, Dysaphis spp, Empoasca spp., Eriosoma larigerum, Erythroneura spp., Gascardia spp., Glycaspis brimblecombei, Hyadaphis pseudobrassicae, Hyalopterus spp, Hyperomyzus pallidus, Idioscopus clypealis, Jacobiasca lybica, Laodelphax spp., Lecanium corni, Lepidosaphes spp., Lopaphis erysimi, Lyogenys maidis, Macrosiphum spp., Mahanarva spp, Metcalfa pruinosa, Metopolophium dirhodum, Myndus crudus, Myzus spp., Neotoxoptera sp, Nephotettix spp., Nilaparvata spp., Nippolachnus piri Mats, Odonaspis ruthae, Oregma lanigera Zehnter, Parabemisia myricae, Paratrioza cockerelli, Parlatoria spp., Pemphigus spp., Peregrinus maidis, Perkinsiella spp, Phorodon humuli, Phylloxera spp, Planococcus spp., Pseudaulacaspis spp., Pseudococcus spp., Pseudatomoscelis seriatus, Psylla spp., Pulvinaria aethiopica, Quadraspidiotus spp., Quesada gigas, Recilia dorsalis, Rhopalosiphum spp., Saissetia spp., Scaphoideus spp., Schizaphis spp., Sitobion spp., Sogatella furcifera, Spissistilus festinus, Tarophagus Proserpina, Toxoptera spp, Trialeurodes spp, Tridiscus sporoboli, Trionymus spp, Trioza erytreae, Unaspis citri, Zygina flammigera, Zyginidia scutellaris;
from the order Hymenoptera, for example, Acromyrmex, Arge spp, Atta spp., Cephus spp., Diprion spp., Diprionidae, Gilpinia polytoma, Hoplocampa spp., Lasius spp., Monomorium pharaonis, Neodiprion spp., Pogonomyrmex spp, Slenopsis invicta, Solenopsis spp. and Vespa spp.;
from the order Isoptera, for example, Coptotermes spp, Corniternes cumulans, Incisitermes spp, Macrotermes spp, Mastotermes spp, Microtermes spp, Reticulitermes spp.; Solenopsis geminate
from the order Lepidoptera, for example, Acleris spp., Adoxophyes spp., Aegeria spp., Agrotis spp., Alabama argillaceae, Amylois spp., Anticarsia gemmatalis, Archips spp., Argyresthia spp, Argyrotaenia spp., Autographa spp., Bucculatrix thurberiella, Busseola fusca, Cadra cautella, Carposina nipponensis, Chilo spp., Choristoneura spp., Chrysoteuchia topiaria, Clysia ambiguella, Cnaphalocrocis spp., Cnephasia spp., Cochylis spp., Coleophora spp., Colias lesbia, Cosmophila flava, Crambus spp, Crocidolomia binotalis, Cryptophlebia leucotreta, Cydalima perspectalis, Cydia spp., Diaphania perspectalis, Diatraea spp., Diparopsis castanea, Earias spp., Elasmopalpus lignosellus, Eldana saccharina, Ephestia spp., Epinotia spp, Estigmene acrea, Etiella zinckinella, Eucosma spp., Eupoecilia ambiguella, Euproctis spp., Euxoa spp., Feltia jaculiferia, Grapholita spp., Hedya nubiferana, Heliothis spp., Hellula undalis, Herpetogramma spp, Hyphantria cunea, Keiferia lycopersicella, Lasmopalpus lignosellus, Leucoptera scitella, Lithocollethis spp., Lobesia botrana, Loxostege bifidalis, Lymantria spp., Lyonetia spp., Malacosoma spp., Mamestra brassicae, Manduca sexta, Mythimna spp, Noctua spp, Operophtera spp., Orniodes indica, Ostrinia nubilalis, Pammene spp., Pandemis spp., Panolis flammea, Papaipema nebris, Pectinophora gossypiela, Perileucoptera coffeella, Pseudaletia unipuncta, Phthorimaea operculella, Pieris rapae, Pieris spp., Plutella xylostella, Prays spp., Pseudoplusia spp, Rachiplusia nu, Richia albicosta, Scirpophaga spp., Sesamia spp., Sparganothis spp., Spodoptera spp., Sylepta derogate, Synanthedon spp., Thaumetopoea spp., Tortrix spp., Trichoplusia ni, Tuta absoluta, and Yponomeuta spp.;
from the order Mallophaga, for example, Damalinea spp. and Trichodectes spp.;
from the order Orthoptera, for example, Blatta spp., Blattella spp., Gryllotalpa spp., Leucophaea maderae, Locusta spp., Neocurtilla hexadactyla, Periplaneta spp., Scapteriscus spp, and Schistocerca spp.;
from the order Psocoptera, for example, Liposcelis spp.;
from the order Siphonaptera, for example, Ceratophyllus spp., Ctenocephalides spp. and Xenopsylla cheopis;
from the order Thysanoptera, for example, Calliothrips phaseoli, Frankliniella spp., Heliothrips spp, Hercinothrips spp., Parthenothrips spp, Scirtothrips aurantii, Sericothrips variabilis, Taeniothrips spp., Thrips spp;
from the order Thysanura, for example, Lepisma saccharina.

Pesticides are herein understood to mean substances that are able to control pest infestation, or occurrence of pests. The composition according to the invention can thus be considered to function as a pesticide. Pesticides comprise insecticides, herbicides, nematicides, molluscicides, fungicides and bactericides. Pesticides can for example be in the form of peptides, proteins, microbial agents or chemical pesticides.

Pests are herein understood to mean organisms that cause harm to other organisms, in particular to agricultural crops. To cause harm comprises to kill, to destroy (parts of), to feed upon, to retard or prevent growth, to reduce physical fitness and to inhibit reproduction.

The insecticidal peptide compositions, or the individual peptides if applied separately or simultaneously, according to the invention can be used in combination with other compounds, including other pesticides such as insecticides, fungicides, or agents that enhance the activity of the composition according to the invention, in for example chemical treatment or pest control programs. The combination may have further surprising advantages, which could be described as synergistic effects.

Suitable other compounds are, for example, compounds of the following classes of active ingredients: organophosphates, nitrophenol derivatives, thioureas, juvenile hormones, formamidines, benzophenone derivatives, ureas, pyrrole derivatives, carbamates, pyrethroids, chlorinated hydrocarbons, acylureas, pyridylmethyleneamino derivatives, macrolides, benzoylureas, neonicotinoids and biological agents such as Bacillus thurigiensis strains or bacterially-derived pesticides such as spinosads, avermectins and Cry proteins.

The insecticidal peptide compositions, or the individual peptides if applied separately or simultaneously, according to the invention can be used in any ratio in combination with other compounds, including other pesticides, insecticides, fungicides or agents that enhance the activity of the peptides according to the invention. Certain ratios are more preferred. A ratio of the insecticidal peptide compositions according to the invention to other compounds of from 50:1 to 1:50 is herein understood to mean that the total amount of molecules of the insecticidal peptides according to the invention is from 50 fold to 1/50^{th} of the other compound. Accordingly, the combination has a ratio of the insecticidal peptide compositions according to the invention to other compounds of from 100:1 to 1:100, from 100:1 to 1:50, from 100:1 to 1:10, from 100:1 to 1:1, from 50:1 to 1:100, from 50:1 to 1:50, from 50:1 to 1:10, from 50:1 to 1:1, from 100:1 to 10:1, from 50:1 to 10:1, from 10:1 to 1:100, from 10:1 to 1:50, from 10:1 to 1:10, from 10:1 to 1:1, from 5:1 to 1:1, from 5:1 to 1:5, from 1:1 to 1:100, from 1:1 to 1:50, from 1:1 to 1:10, from 1:1 to 1:5, from 1:10 to 1:50, from 1:10 to 1:100 or from 1:50 to 1:100. Preferably, the ratio is from 10:1 to 1:100, from 10:1 to 1:50, from 10:1 to 1:10, from 1:1 to 1:100, from 1:1 to 1:50, from 5:1 to 1:5 or from 1:1 to 1:10.

The concentrations of the different active ingredients in the compositions according to the invention in combination with other compounds can vary depending on the specific formulation, environmental conditions, methods of application and the extent of pesticidal activity of the individual components.

Plants and Crops:

The active ingredients according to the invention can be used for controlling, i.e. containing or destroying, pests of the abovementioned type which occur in particular on plants, especially on useful plants and ornamentals in agriculture, in horticulture and in forests, or on organs, such as fruits, flowers, foliage, stalks, tubers or roots, of such plants, and in some cases even plant organs which are formed at a later point in time remain protected against these pests.

Suitable target crops are, in particular, cereals, such as wheat, barley, rye, oats, rice, maize or sorghum; beet, such as sugar or fodder beet; fruit, for example pomaceous fruit, stone fruit or soft fruit, such as apples, pears, plums, peaches, almonds, cherries or berries, for example strawberries, raspberries or blackberries; leguminous crops, such as beans, lentils, peas or soya; oil crops, such as oilseed rape, mustard, poppies, olives, sunflowers, coconut, castor, cocoa or ground nuts; cucurbits, such as pumpkins, cucumbers or melons; fibre plants, such as cotton, flax, hemp orjute; citrus fruit, such as oranges, lemons, grapefruit or tangerines; vegetables, such as spinach, lettuce, asparagus, cabbages, carrots, onions, tomatoes, potatoes or bell peppers; Lauraceae, such as avocado, Cinnamonium or camphor; and also tobacco, nuts, coffee, eggplants, sugarcane, tea, pepper, grapevines, hops, the plantain family and latex plants.

The compositions and/or methods of the present invention may be also used on any ornamental and/or vegetable crops, including flowers, shrubs, broad-leaved trees and evergreens.

For example the invention may be used on any of the following ornamental species: Ageratum spp., Alonsoa spp., Anemone spp., Anisodontea capsenisis, Anthemis spp., Antirrhinum spp., Aster spp., Begonia spp. (e.g. B. elatior, B. semperflorens, B. tubereux), Bougainvillea spp., Brachycome spp., Brassica spp. (ornamental), Calceolaria spp., Capsicum annuum, Catharanthus roseus, Canna spp., Centaurea spp., Chrysanthemum spp., Cineraria spp. (C. maritime), Coreopsis spp., Crassula coccinea, Cuphea ignea, Dahlia spp., Delphinium spp., Dicentra spectabilis, Dorotheantus spp., Eustoma grandiflorum, Forsythia spp., Fuchsia spp., Geranium gnaphalium, Gerbera spp., Gomphrena globosa, Heliotropium spp., Helianthus spp., Hibiscus spp., Hortensia spp., Hydrangea spp., Hypoestes phyllostachya, Impatiens spp. (I. Walleriana), Iresines spp., Kalanchoe spp., Lantana camara, Lavatera trimestris, Leonotis leonurus, Lilium spp., Mesembryanthemum spp., Mimulus spp., Monarda spp., Nemesia spp., Tagetes spp., Dianthus spp. (carnation), Canna spp., Oxalis spp., Bellis spp., Pelargonium spp. (P. peltatum, P. zonale), Viola spp. (pansy), Petunia spp., Phlox spp., Plecthranthus spp., Poinsettia spp., Parthenocissus spp. (P. quinquefolia, P. tricuspidata), Primula spp., Ranunculus spp., Rhododendron spp., Rosa spp. (rose), Rudbeckia spp., Saintpaulia spp., Salvia spp., Scaevola aemola, Schizanthus wisetonensis, Sedum spp., Solanum spp., Surfinia spp., Tagetes spp., Nicotinia spp., Verbena spp., Zinnia spp. and other bedding plants.

For example the invention may be used on any of the following vegetable species: Allium spp. (A. sativum, A. cepa, A. oschaninii, A. Porrum, A. ascalonicum, A. fistulosum), Anthriscus cerefolium, Apium graveolus, Asparagus officinalis, Beta vulgarus, Brassica spp. (B. Oleracea, B. Pekinensis, B. rapa), Capsicum annuum, Cicer arietinum, Cichorium endivia, Cichorum spp. (C. intybus, C. endivia), Citrillus lanatus, Cucumis spp. (C. sativus, C. melo), Cucurbita spp. (C. pepo, C. maxima), Cyanara spp. (C. scolymus, C. cardunculus), Daucus carota, Foeniculum vulgare, Hypericum spp., Lactuca sativa, Lycopersicon spp. (L. esculentum, L. lycopersicum), Mentha spp., Ocimum basilicum, Petroselinum crispum, Phaseolus spp. (P. vulgaris, P. coccineus), Pisum sativum, Raphanus sativus, Rheum rhaponticum, Rosemarinus spp., Salvia spp., Scorzonera hispanica, Solanum melongena, Spinacea oleracea, Valerianella spp. (V. locusta, V. eriocarpa) and Vicia faba.

Preferred ornamental species include African violet, Begonia, Dahlia, Gerbera, Hydrangea, Verbena, Rosa, Kalanchoe, Poinsettia, Aster, Centaurea, Coreopsis, Delphinium, Monarda, Phlox, Rudbeckia, Sedum, Petunia, Viola, Impatiens, Geranium, Chrysanthemum, Ranunculus, Fuchsia, Salvia, Hortensia, rosemary, sage, St. Johnswort, mint, sweet pepper, tomato and cucumber.

The compositions according to the invention are especially suitable for controlling Aphis craccivora, Diabrotica balteata, Heliothis virescens, Myzus persicae, Plutella xylostella and Spodoptera littoralis in cotton, vegetable, maize, rice and soya crops. The active ingredients according to the invention are further especially suitable for controlling Mamestra (preferably in vegetables), Cydia pomonella (preferably in apples), Empoasca (preferably in vegetables, vineyards), Leptinotarsa (preferably in potatos) and Chilo supressalis (preferably in rice).

The compositions according to the first aspect are particularly suitable for control of:
a pest of the order Hemiptera, for example, one or more of the species Bemisia tabaci, Aphis craccivora, Myzus persicae, Rhopalosiphum Padi, Nilaparvata lugens, and Euschistus heros (preferably in vegetables, soybeans, and sugarcane);
a pest of the order Lepidoptera, for example, one or more of the species Spodoptera littoralis, Spodoptera frugiperda, Plutella xylostella, Cnaphalocrocis medinalis, Cydia pomonella, Chrysodeixis includes, Chilo suppressalis, Elasmopalpus lignosellus, Pseudoplusia includens, and Tuta absoluta (preferably in vegetables and corn);
a pest of the order Thysanoptera, such as the family Thripidae, for example, one or more of Thrips tabaci and Frankliniella occidentalis (preferably in vegetables); and
soil pests (such as of the order Coleoptera), for example, the species Diabrotica balteata, Agriotes spp. and Leptinotarsa decemlineata (preferably in vegetables and corn).

The insecticidal peptide compositions of the invention are especially suitable for control of one or more of pests selected from mites, thrips, whiteflies, aphids, psyllids, fruit flies, loopers, bollworms, and budworms; in particular for fruit and vegetable crops, whether in greenhouse or field.

The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins, for example insecticidal proteins from Bacillus cereus or Bacillus popilliae; or insecticidal proteins from Bacillus thuringiensis, such as δ-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

The processes for the preparation of such transgenic plants are generally known to the person skilled in the art and are described, for example, in the publications mentioned above. Cryl-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and moths (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II® (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:
1. Bt11 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.
2. Bt176 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.
3. MIR604 Maize from Syngenta Seeds SAS, Chemin de I'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.
4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.
5. IPC 531 Cotton from Monsanto Europe S.A. 270-272 Avenue de Tervuren, B-1150 Brussels, Belgium, registration number C/ES/96/02.
6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.
7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

Transgenic crops of insect-resistant plants are also described in BATS (Zentrum für Biosicherheit und Nachhaltigkeit, Zentrum BATS, Clarastrasse 13, 4058 Basel, Switzerland) Report 2003, (http://bats.ch).

The term “crops” herein is to be understood as to also include crop plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818 and EP-A-0 353 191. The methods of producing such transgenic plants are generally known to the person skilled in the art and are described, for example, in the publications mentioned above.

Crops may also be modified for enhanced resistance to fungal (for example Fusarium, Anthracnose, or Phytophthora), bacterial (for example Pseudomonas) or viral (for example potato leafroll virus, tomato spotted wilt virus, cucumber mosaic virus) pathogens.

Crops also include those that have enhanced resistance to nematodes, such as the soybean cyst nematode.

Crops that are tolerance to abiotic stress include those that have enhanced tolerance to drought, high salt, high temperature, chill, frost, or light radiation, for example through expression of NF-YB or other proteins known in the art.

Antipathogenic substances which can be expressed by such transgenic plants include, for example, ion channel blockers, such as blockers for sodium and calcium channels, for example the viral KP1, KP4 or KP6 toxins; stilbene synthases; bibenzyl synthases; chitinases; glucanases; the so-called “pathogenesis-related proteins” (PRPs; see e.g. EP-A-0 392 225); antipathogenic substances produced by microorganisms, for example peptide antibiotics or heterocyclic antibiotics (see e.g. WO 95/33818) or protein or polypeptide factors involved in plant pathogen defence (so-called “plant disease resistance genes”, as described in WO 03/000906).

Further areas of use of the compositions according to the invention are the protection of stored goods and store rooms and the protection of raw materials, such as wood, textiles, floor coverings or buildings, and also in the hygiene sector, especially the protection of humans, domestic animals and productive livestock against pests of the mentioned type.

The present invention may also provide a composition of the first aspect, for use in controlling parasites in or on an animal. The present invention further provides a composition of the first aspect, for use in controlling ectoparasites on an animal. The present invention further provides a composition of the first aspect, for use in preventing and/or treating diseases transmitted by ectoparasites.

The present invention may also provide the use of a composition the first aspect, for the manufacture of a medicament for controlling parasites in or on an animal. The present invention further provides the use of a compound of the first aspect, for the manufacture of a medicament for controlling ectoparasites on an animal. The present invention further provides the use of a compound of the first aspect, for the manufacture of a medicament for preventing and/or treating diseases transmitted by ectoparasites.

A “parasite” is a pest which lives in or on the host animal and benefits by deriving nutrients at the host animal's expense. An “endoparasite” is a parasite which lives in the host animal. An “ectoparasite” is a parasite which lives on the host animal. Ectoparasites include, but are not limited to, acari, insects and crustaceans (e.g. sea lice). The Acari (or Acarina) sub-class comprises ticks and mites. Ticks include, but are not limited to, members of the following genera: Rhipicaphalus, for example, Rhipicaphalus (Boophilus) microplus and Rhipicephalus sanguineus; Amblyomrna; Dermacentor; Haemaphysalis; Hyalomma; Ixodes; Rhipicentor; Margaropus; Argas; Otobius; and Ornithodoros. Mites include, but are not limited to, members of the following genera: Chorioptes, for example Chorioptes bovis; Psoroptes, for example Psoroptes ovis; Cheyletiella; Dermanyssus; for example Dermanyssus gallinae; Ortnithonyssus; Demodex, for example Demodex canis; Sarcoptes, for example Sarcoptes scabiei; and Psorergates. Insects include, but are not limited to, members of the orders: Siphonaptera, Diptera, Phthiraptera, Lepidoptera, Coleoptera and Homoptera. Members of the Siphonaptera order include, but are not limited to, Ctenocephalides felis and Ctenocephatides canis. Members of the Diptera order include, but are not limited to, Musca spp.; bot fly, for example Gasterophilus intestinalis and estrus ovis; biting flies; horse flies, for example Haematopota spp. and Tabunus spp.; haematobia, for example haematobia irritans; Stomoxys; Lucilia; midges; and mosquitoes. Members of the Phthiraptera class include, but are not limited to, blood sucking lice and chewing lice, for example Bovicola Ovis and Bovicola Bovis.

Further areas of use of the compositions according to the invention are the field of tree injection/trunk treatment for all ornamental trees as well all sort of fruit and nut trees.

In the field of tree injection/trunk treatment, the compounds according to the present invention are especially suitable against wood-boring insects from the order Lepidoptera as mentioned above and from the order Coleoptera, especially against woodborers listed in the following tables X and Y:

TABLE X
Examples of exotic woodborers of economic importance.

| Family | Species | Host or Crop Infested |
|---|---|---|
| Buprestidae | Agrilus planipennis | Ash |
| Cerambycidae | Anoplura glabripennis | Hardwoods |
| Scolytidae | Xylosandrus crassiusculus | Hardwoods |
| | X. mutilatus | Hardwoods |
| | Tomicus piniperda | Conifers |

TABLE Y
Examples of native woodborers of economic importance.

| Family | Species | Host or Crop Infested |
|---|---|---|
| Buprestidae | Agrilus anxius | Birch |
| | Agrilus politus | Willow, Maple |
| | Agrilus sayi | Bayberry, Sweetfern |
| | Agrilus vittaticolllis | Apple, Pear, Cranberry, Serviceberry, |
| | | Hawthorn |
| | Chrysobothris femorata | Apple, Apricot, Beech, Boxelder, Cherry, |
| | | Chestnut, Currant, Elm, Hawthorn, |
| | | Hackberry, Hickory, Horsechestnut, |
| | | Linden, Maple, Mountain-ash, Oak, |
| | | Pecan, Pear, Peach, Persimmon, Plum, |
| | | Poplar, Quince, Redbud, Serviceberry, |
| | | Sycamore, Walnut, Willow |
| | Texania campestris | Basswood, Beech, Maple, Oak, |
| | | Sycamore, Willow, Yellow-poplar |
| Cerambycidae | Goes pulverulentus | Beech, Elm, Nuttall, Willow, Black oak, |
| | | Cherrybark oak, Water oak, Sycamore |
| | Goes tigrinus | Oak |
| | Neoclytus acuminatus | Ash, Hickory, Oak, Walnut, Birch, Beech, |
| | | Maple, Eastern hophornbeam, Dogwood, |
| | | Persimmon, Redbud, Holly, Hackberry, |
| | | Black locust, Honeylocust, Yellow-poplar, |
| | | Chestnut, Osage-orange, Sassafras, |
| | | Lilac, Mountain-mahogany, Pear, Cherry, |
| | | Plum, Peach, Apple, Elm, Basswood, |
| | | Sweetgum |
| | Neoptychodes trilineatus | Fig, Alder, Mulberry, Willow, Netleaf |
| | | hackberry |
| | Oberea ocellata | Sumac, Apple, Peach, Plum, Pear, |
| | | Currant, Blackberry |
| | Oberea tripunctata | Dogwood, Viburnum, Elm, Sourwood, |
| | | Blueberry, Rhododendron, Azalea, Laurel, |
| | | Poplar, Willow, Mulberry |
| | Oncideres cingulata | Hickory, Pecan, Persimmon, Elm, |
| | | Sourwood, Basswood, Honeylocust, |
| | | Dogwood, Eucalyptus, Oak, Hackberry, |
| | | Maple, Fruit trees |
| | Saperda calcarata | Poplar |
| | Strophiona nitens | Chestnut, Oak, Hickory, Walnut, Beech, |
| | | Maple |
| Scolytidae | Corthylus columbianus | Maple, Oak, Yellow-poplar, Beech, |
| | | Boxelder, Sycamore, Birch, Basswood, |
| | | Chestnut, Elm |
| | Dendroctonus frontalis | Pine |
| | Dryocoetes betulae | Birch, Sweetgum, Wild cherry, Beech, |
| | | Pear |
| | Monarthrum fasciatum | Oak, Maple, Birch, Chestnut, Sweetgum, |
| | | Blackgum, Poplar, Hickory, Mimosa, |
| | | Apple, Peach, Pine |
| | Phloeotribus liminaris | Peach, Cherry, Plum, Black cherry, Elm, |
| | | Mulberry, Mountain-ash |
| | Pseudopityophthorus pruinosus | Oak, American beech, Black cherry, |
| | | Chickasaw plum, Chestnut, Maple, |
| | | Hickory, Hornbeam, Hophornbeam |
| Sesiidae | Paranthrene simulans | Oak, American chestnut |
| | Sannina uroceriformis | Persimmon |
| | Synanthedon exitiosa | Peach, Plum, Nectarine, Cherry, Apricot, |
| | | Almond, Black cherry |
| | Synanthedon pictipes | Peach, Plum, Cherry, Beach, Black |
| | | Cherry |
| | Synanthedon rubrofascia | Tupelo |
| | Synanthedon scitula | Dogwood, Pecan, Hickory, Oak, Chestnut, |
| | | Beech, Birch, Black cherry, Elm, |
| | | Mountain-ash, Viburnum, Willow, Apple, |
| | | Loquat, Ninebark, Bayberry |
| | Vitacea polistiformis | Grape |

The present invention may be also used to control any pests that may be present in turfgrass, including for example beetles, caterpillars, fire ants, ground pearls, millipedes, sow bugs, mites, mole crickets, scales, mealybugs, ticks, spittlebugs, southern chinch bugs and white grubs. The present invention may be used to control pests at various stages of their life cycle, including eggs, larvae, nymphs and adults.

In particular, the present invention may be used to control pests that feed on the roots of turfgrass including white grubs (such as Cyclocephala spp. (e.g. masked chafer, C. lurida), Rhizotrogus spp. (e.g. European chafer, R. majalis), Cotinus spp. (e.g. Green June beetle, C. nitida), Popillia spp. (e.g. Japanese beetle, P. japonica), Phyllophaga spp. (e.g. May/June beetle), Ataenius spp. (e.g. Black turfgrass ataenius, A. spretulus), Maladera spp. (e.g. Asiatic garden beetle, M. castanea) and Tomarus spp.), ground pearls (Margarodes spp.), mole crickets (tawny, southern, and short-winged; Scapteriscus spp., Gryllotalpa africana) and leatherjackets (European crane fly, Tipula spp.).

The present invention may also be used to control pests of turfgrass that are thatch dwelling, including armyworms (such as fall armyworm Spodoptera frugiperda, and common armyworm Pseudaletia unipuncta), cutworms, billbugs (Sphenophorus spp., such as S. venatus verstitus and S. parvulus), and sod webworms (such as Crambus spp. and the tropical sod webworm, Herpetogramma phaeopteralis).

The present invention may also be used to control pests of turfgrass that live above the ground and feed on the turfgrass leaves, including chinch bugs (such as southern chinch bugs, Blissus insularis), Bermudagrass mite (Eriophyes cynodoniensis), rhodesgrass mealybug (Antonina graminis), two-lined spittlebug (Propsapia bicincta), leafhoppers, cutworms (Noctuidae family), and greenbugs.

The present invention may also be used to control other pests of turfgrass such as red imported fire ants (Solenopsis invicta) that create ant mounds in turf.

In the hygiene sector, the compositions according to the invention are active against ectoparasites such as hard ticks, soft ticks, mange mites, harvest mites, flies (biting and licking), parasitic fly larvae, lice, hair lice, bird lice and fleas.

Examples of such parasites are:
Of the order Anoplurida: Haematopinus spp., Linognathus spp., Pediculus spp. and Phtirus spp., Solenopotes spp.
Of the order Mallophagida: Trimenopon spp., Menopon spp., Trinoton spp., Bovicola spp., Werneckiella spp., Lepikentron spp., Damalina spp., Trichodectes spp. and Felicola spp.
Of the order Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.
Of the order Siphonapterida, for example Pulex spp., Ctenocephalides spp., Xenopsylla spp., Ceratophyllus spp.
Of the order Heteropterida, for example Cimex spp., Triatoma spp., Rhodnius spp., Panstrongylus spp.
Of the order Blattarida, for example Blatta orientalis, Periplaneta americana, Blattelagermanica and Supella spp.
Of the subclass Acaria (Acarida) and the orders Meta- and Meso-stigmata, for example Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.
Of the orders Actinedida (Prostigmata) and Acaridida (Astigmata), for example Acarapis spp., Cheyletiella spp., Ornithocheyletia spp., Myobia spp., Psorergatesspp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The compositions according to the invention can be used, for example, against the following pests: beetles such as Hylotrupes bajulus, Chlorophorus pilosis, Anobium punctatum, Xestobium rufovillosum, Ptilinuspecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthesrugicollis, Xyleborusspec., Tryptodendron spec., Apate monachus, Bostrychus capucins, Heterobostrychus brunneus, Sinoxylon spec. and Dinoderus minutus, and also hymenopterans such as Sirex juvencus, Urocerus gigas, Urocerus gigas taignus and Urocerus augur, and termites such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis and Coptotermes formosanus, and bristletails such as Lepisma saccharina.

The compositions according to the invention are especially suitable for controlling one or more pests selected from the family: Noctuidae, Plutellidae, Chrysomelidae, Thripidae, Pentatomidae, Tortricidae, Delphacidae, Aphididae, Noctuidae, Crambidae, Meloidogynidae, and Heteroderidae.

The composition according to the invention are especially suitable for controlling one or more of pests selected from the genus: Spodoptera spp, Plutella spp, Frankliniella spp, Thrips spp, Euschistus spp, Cydia spp, Nilaparvata spp, Myzus spp, Aphis spp, Diabrotica spp, Rhopalosiphum spp, Pseudoplusia spp and Chilo spp.

The compositions are especially suitable for controlling one or more of Spodoptera littoralis, Plutella xylostella, Frankliniella occidentalis, Thrips tabaci, Euschistus heros, Cydia pomonella, Nilaparvata lugens, Myzus persicae, Chrysodeixis includens, Aphis craccivora, Diabrotica balteata, Rhopalosiphum padi, and Chilo suppressalis.

In an embodiment, of each aspect, a composition according to the invention is suitable for controlling Spodoptera littoralis, Plutella xylostella, Frankliniella occidentalis, Thrips tabaci, Euschistus heros, Cydia pomonella, Nilaparvata lugens, Myzus persicae, Chrysodeixis includens, Aphis craccivora, Diabrotica balteata, Rhopalosiphum Padia, and Chilo Suppressalis in cotton, vegetable, maize, cereal, rice and soya crops.

In an embodiment, a composition according to the invention is suitable for controlling Mamestra (preferably in vegetables), Cydia pomonella (preferably in apples), Empoasca (preferably in vegetables, vineyards), Leptinotarsa (preferably in potatos) and Chilo supressalis (preferably in rice).

### EXAMPLES

The following biological examples serve to illustrate the invention and are non-limitative.

### Example 1: Increased Effect of Hv1a and kB1 Against Spodoptera littoralis L1

Gossypium hirsutum leaf discs with a 5 cm diameter were cut out and placed in a Petri dish which was prepared with 3 filter paper and 800 μl water. 100 μl of test solution was applied on the lower surface by spraying and dried to air. Next, the leaf was flipped and 100 μl of test solution was applied on the upper surface by spraying and dried to air. The leaf was infested with 10 L1 larvae of Spodoptera littoralis, after which the dish was closed with a cotton filter and a plastic lid. The Petri dish was placed in a climate chamber at a temperature of 25° C., a relative humidity of 65% and with no artificial light cycle. Two days after infestation 200 μl water was added to the Petri dish. Four days after infestation 500 μl water was added to the Petri dish. Three Petri dishes were assessed per treatment. Results of observed mortality are shown in Table A below. All observed responses are corrected for control mortality using Abbott's formula.

TABLE A
observed mortality of Spodoptera littoralis L1

| | 1 d after | 2 d after | 3 d after | 6 d after |
|---|---|---|---|---|
| | infesta- | infesta- | infesta- | infesta- |
| Test solution | tion | tion | tion | tion |
| Control (Acetonitrile 5%) | 7% | 13% | 20% | 17% |
| SEQ ID NO: 3 (Hv1a) | 0% | 0% | 0% | 0% |
| (10000 ppm) | | | | |
| SEQ ID NO: 1 (kB1) | 0% | 35% | 29% | 32% |
| (10000 ppm) | | | | |
| SEQ ID NO: 3 (Hv1a) | 68% | 96% | 96% | 96% |
| (10000 ppm) + | | | | |
| SEQ ID NO: 1 (kB1) | | | | |
| (10000 ppm) | | | | |

kB1 is cyclotide kalata B1;
Hv1a is ω-hexatoxin peptide;

### Example 2: Increased Effect of Hv1a and kB1 Against Plutella xylostella L1

Brassica chinensis leaf discs with a 5 cm diameter were cut out and placed in a Petri dish which was prepared with 3 filter paper and 800 μl water. 100 μl of test solution was applied on the lower surface by spraying and dried to air. Next, the leaf was flipped and 100 μl of test solution was applied on the upper surface by spraying and dried to air. The leaf 3 was infested with 10 L1 larvae of Plutella xylostella, after which the dish was closed with a cotton filter and a plastic lid. The Petri dish was placed in a climate chamber at a temperature of 25° C., a relative humidity of 65% and with no artificial light cycle. Four days after infestation 150 μl water was added to the Petri dish. Results of observed mortality are shown in Tables B1 & B2 below. Three Petri dishes were assessed per treatment. All observed responses are corrected for control mortality using Abbott's formula.

TABLE B1
observed mortality of Plutella xylostella L1

| | 1 d after | 2 d after | 3 d after | 4 d after | 7 d after |
|---|---|---|---|---|---|
| | infesta- | infesta- | infesta- | infesta- | infesta- |
| Test solution | tion | tion | tion | tion | tion |
| Control (Acetonitrile 5%) | 0% | 3% | 3% | 3% | 3% |
| SEQ ID NO: 3 (Hv1a) | 0% | 7% | 10% | 7% | 21% |
| (625 ppm) | | | | | |
| SEQ ID NO: 1 (kB1) | 0% | 0% | 3% | 14% | 17% |
| (625 ppm) | | | | | |
| SEQ ID NO: 3 (Hv1a) | 0% | 72% | 93% | 93% | 93% |
| (625 ppm) + | | | | | |
| SEQ ID NO: 1 (kB1) | | | | | |
| (625 ppm) | | | | | |

kB1 is cyclotide kalata B1;
Hv1a is ω-hexatoxin peptide;

TABLE B2
observed mortality of Plutella xylostella L1

| | 1 d after | 2 d after | 3 d after | 4 d after | 7 d after |
|---|---|---|---|---|---|
| | infesta- | infesta- | infesta- | infesta- | infesta- |
| Test solution | tion | tion | tion | tion | tion |
| Control (Acetonitrile 5%) | 10% | 10% | 23% | 23% | 33% |
| SEQ ID NO: 3 (Hv1a) | 0% | 15% | 26% | 22% | 25% |
| (625 ppm) | | | | | |
| SEQ ID NO: 2 (kB2) | 0% | 0% | 0% | 0% | 0% |
| (157 ppm) | | | | | |
| SEQ ID NO: 3 (Hv1a) | 26% | 48% | 70% | 70% | 70% |
| (625 ppm) + | | | | | |
| SEQ ID NO: 2 (kB2) | | | | | |
| (157 ppm) | | | | | |

kB1 is cyclotide kalata B1;
Hv1a is ω-hexatoxin peptide;

### Example 3: Increased Effect of Peptide Toxins Hv1a, Av3, GS-ω/κ-Hxtx-Hv1h and Cyclotides kB1 and kB2 Against Plutella xylostella L1

Brassica chinensis leaf discs with a 5 cm diameter were cut out and placed in a Petri dish which was prepared with 3 filter paper and 800 μl water. 100 μl of test solution was applied on the lower surface by spraying and dried to air. Next, the leaf was flipped and 100 μl of test solution was applied on the upper surface by spraying and dried to air. The leaf 3 was infested with 10 L1 larvae of Plutella xylostella, after which the dish was closed with a cotton filter and a plastic lid. The Petri dish was placed in a climate chamber at a temperature of 25° C., a relative humidity of 65% and with no artificial light cycle. Four days after infestation 150 μl water was added to the Petri dish. Results of observed mortality are shown in Table C below. Three Petri dishes were assessed per treatment. All observed responses are corrected for control mortality using Abbott's formula.

TABLE C
observed mortality of Plutella xylostella L1

| | 1 d after | 2 d after | 3 d after | 4 d after | 6 d after |
|---|---|---|---|---|---|
| | infesta- | infesta- | infesta- | infesta- | infesta- |
| Test solution | tion | tion | tion | tion | tion |
| Control (Acetonitrile 5%) | 10% | 10% | 23% | 23% | 33% |
| SEQ ID NO: 3 (Hv1a) | 0% | 15% | 26% | 22% | 25% |
| (625 ppm) | | | | | |
| SEQ ID NO: 4 (Av3) | 0% | 15% | 13% | 17% | 10% |
| (2500 ppm) | | | | | |
| SEQ ID NO: 5 | 0% | 0% | 0% | 0% | 0% |
| (GS-ω/κ-Hxtx-Hv1h) | | | | | |
| (2500 ppm) | | | | | |
| SEQ ID NO: 1 (kB1) | 0% | 11% | 9% | 22% | 20% |
| (625 ppm) | | | | | |
| SEQ ID NO: 6 | 0% | 0% | 0% | 0% | 0% |
| (kB1[V25K]) | | | | | |
| (625 ppm) | | | | | |
| SEQ ID NO: 2 (kB2) | 0% | 0% | 4% | 4% | 0% |
| (625 ppm) | | | | | |
| SEQ ID NO: 3 (Hv1a) | 70% | 93% | 96% | 96% | 100% |
| (625 ppm) + | | | | | |
| SEQ ID NO: 2 (kB2) | | | | | |
| (625 ppm) | | | | | |
| SEQ ID NO: 3 (Hv1a) | 15% | 56% | 61% | 74% | 75% |
| (625 ppm) + | | | | | |
| SEQ ID NO: 6 | | | | | |
| (kB1[V25K] | | | | | |
| (625 ppm) | | | | | |
| SEQ ID NO: 4 (Av3) | 48% | 93% | 96% | 100% | 100% |
| (2500 ppm) + | | | | | |
| SEQ ID NO: 1 (kB1) | | | | | |
| (625 ppm) | | | | | |
| SEQ ID NO: 5 | 15% | 81% | 96% | 100% | 100% |
| (GS-ω/κ-Hxtx-Hv1h) | | | | | |
| (2500 ppm) + | | | | | |
| SEQ ID NO: 1 (kB1) | | | | | |
| (625 ppm) | | | | | |

### Example 4: Increased Effect of Hv1a and kB1 Against Plutella xylostella L3

Brassica chinensis leaf discs with a 5 cm diameter were cut out and placed in a Petri dish which was prepared with 3 filter paper and 800 μl water. 100 μl of test solution was applied on the lower surface by spraying and dried to air. Next, the leaf was flipped and 100 μl of test solution was applied on the upper surface by spraying and dried to air. The leaf was infested with 10 L3 larvae (6-7 days old) of Plutella xylostella, after which the dish was closed with a cotton filter and a plastic lid. The Petri dish was placed in a climate chamber at a temperature of 25° C., a relative humidity of 65% and with no artificial light cycle. Four days after infestation 150 μl water was added to the Petri dish. Results of observed mortality are shown in Table 0 below. Three Petri dishes were assessed per treatment. All observed responses are corrected for control mortality using Abbott's formula.

TABLE D
observed mortality of Plutella xylostella L3

| | 1 d after | 2 d after | 3 d after | 4 d after |
|---|---|---|---|---|
| | infesta- | infesta- | infesta- | infesta- |
| Test solution | tion | tion | tion | tion |
| Control (Acetonitrile 5%) | 0% | 0% | 0% | 0% |
| SEQ ID NO: 3 (Hv1a) | 0% | 0% | 0% | 0% |
| (2500 ppm) | | | | |
| SEQ ID NO: 3 (Hv1a) | 0% | 0% | 0% | 0% |
| (5000 ppm) | | | | |
| SEQ ID NO: 1 (kB1) | 0% | 0% | 3% | 20% |
| (2500 ppm) | | | | |
| SEQ ID NO: 1 (kB1) | 0% | 0% | 3% | 37% |
| (5000 ppm) | | | | |
| SEQ ID NO: 3 (Hv1a) | 17% | 73% | 80% | 90% |
| (2500 ppm) + | | | | |
| SEQ ID NO: 1 (kB1) | | | | |
| (2500 ppm) | | | | |
| SEQ ID NO: 3 (Hv1a) | 17% | 73% | 80% | 90% |
| (2500 ppm) + | | | | |
| SEQ ID NO: 1 (kB1) | | | | |
| (5000 ppm) | | | | |
| SEQ ID NO: 3 (Hv1a) | 63% | 90% | 90% | 97% |
| (5000 ppm) + | | | | |
| SEQ ID NO: 1 (kB1) | | | | |
| (2500 ppm) | | | | |
| SEQ ID NO: 3 (Hv1a) | 57% | 97% | 100% | 100% |
| (5000 ppm) + | | | | |
| SEQ ID NO: 1 (kB1) | | | | |
| (5000 ppm) | | | | |

### Example 5: Increased Effect of Hv1a and kB1 Against Cydia pomonella L1

An artificial diet was prepared. 1.5 ml of artificial diet was applied in a well of a 24-well plate followed by 50 μl of test solution by spraying. The well-plate was infested with 1 L1 larva of Cydia pomonella per well, after which the plate was closed with a carton filter and a metal lid. The 24-well plate was placed in a climate chamber at a temperature of 25° C., a relative humidity of 65% and with no artificial light cycle. Results of observed mortality are shown in Table F below. Five wells (replicates) were assessed per treatment. All observed responses are corrected for control mortality using Abbott's formula.

TABLE E
observed mortality of Cydia pomonella L1

| | 1 d after | 2 d after | 3 d after | 4 d after | 7 d after |
|---|---|---|---|---|---|
| | infesta- | infesta- | infesta- | infesta- | infesta- |
| Test solution | tion | tion | tion | tion | tion |
| Control (Acetonitrile 5%) | 0% | 0% | 0% | 0% | 0% |
| SEQ ID NO: 3 (Hv1a) | 0% | 0% | 0% | 0% | 0% |
| (10000 ppm) | | | | | |
| SEQ ID NO: 1 (kB1) | 20% | 20% | 20% | 20% | 20% |
| (10000 ppm) | | | | | |
| SEQ ID NO: 3 (Hv1a) | 60% | 100% | 100% | 100% | 100% |
| (10000 ppm) + | | | | | |
| SEQ ID NO: 1 (kB1) | | | | | |
| (10000 ppm) | | | | | |

### Example 6: Increased Effect of GS-ω/κ-Hxtx-Hv1h and kB1 Against Plutella xylostella L3

Brassica chinensis leaf discs with a 5 cm diameter were cut out and placed in a Petri dish which was prepared with 3 filter paper and 800 μl water. 100 μl of test solution was applied on the lower surface by spraying and dried to air. Next, the leaf was flipped and 100 μl of test solution was applied on the upper surface by spraying and dried to air. The leaf was infested with 10 L3 larvae (6-7 days old) of Plutella xylostella, after which the dish was closed with a cotton filter and a plastic lid. The Petri dish was placed in a climate chamber at a temperature of 25° C., a relative humidity of 65% and with no artificial light cycle. Four days after infestation 150 μl water was added to the Petri dish. Results of observed mortality are shown in Table 0 below. Three Petri dishes were assessed per treatment. All observed responses are corrected for control mortality using Abbott's formula.

TABLE F
observed mortality of Plutella xylostella L3

| | 1 d after | 2 d after | 3 d after |
|---|---|---|---|
| | infesta- | infesta- | infesta- |
| Test solution | tion | tion | tion |
| Control (Acetonitrile 5%) | 1.7% | 1.7% | 1.7% |
| SEQ ID NO: 5 (GS-ω/κ- | 0% | 0% | 0% |
| Hxtx-Hv1h) (5000 ppm) | | | |
| SEQ ID NO: 5 (GS-ω/κ- | 0% | 0% | 0% |
| Hxtx-Hv1h) (2500 ppm) | | | |
| SEQ ID NO: 5 (GS-ω/κ- | 0% | 0% | 0% |
| Hxtx-Hv1h) (1250 ppm) | | | |
| SEQ ID NO: 5 (GS-ω/κ- | 0% | 0% | 0% |
| Hxtx-Hv1h) (625 ppm) | | | |
| SEQ ID NO: 1 (kB1) | 0% | 0% | 22% |
| (5000 ppm) | | | |
| SEQ ID NO: 5 (GS-ω/κ- | 9% | 83% | 90% |
| Hxtx-Hv1h) (5000 ppm) + | | | |
| SEQ ID NO: 1 (kB1) | | | |
| (5000 ppm) | | | |
| SEQ ID NO: 5 (GS-ω/κ- | 0% | 49% | 70% |
| Hxtx-Hv1h) (2500 ppm) + | | | |
| SEQ ID NO: 1 (kB1) | | | |
| (5000 ppm) | | | |
| SEQ ID NO: 5 (GS-ω/κ- | 0% | 25% | 56% |
| Hxtx-Hv1h) (1250 ppm) + | | | |
| SEQ ID NO: 1 (kB1) | | | |
| (5000 ppm) | | | |
| SEQ ID NO: 5 (GS-ω/κ- | 0% | 42% | 70% |
| Hxtx-Hv1h) (625 ppm) + | | | |
| SEQ ID NO: 1 (kB1) | | | |
| (5000 ppm) | | | |

### Example 7: Increased Effect of GS-ω/κ-Hxtx-Hv1h and kB1 Against Spodoptera littoralis L1

Gossypium hirsutum leaf discs with a 5 cm diameter were cut out and placed in a Petri dish which was prepared with 3 filter paper and 800 μl water. 100 μl of test solution was applied on the lower surface by spraying and dried to air. Next, the leaf was flipped and 100 μl of test solution was applied on the upper surface by spraying and dried to air. The leaf was infested with 10 L1 larvae of Spodoptera littoralis, after which the dish was closed with a cotton filter and a plastic lid. The Petri dish was placed in a climate chamber at a temperature of 25° C., a relative humidity of 65% and with no artificial light cycle. Two days after infestation 200 μl water was added to the Petri dish. Four days after infestation 500 μl water was added to the Petri dish. Three Petri dishes were assessed per treatment. Results of observed mortality are shown in Table A below. All observed responses are corrected for control mortality using Abbott's formula.

TABLE G
observed mortality of Spodoptera littoralis L1

| | 1 d after | 2 d after | 3 d after |
|---|---|---|---|
| | infesta- | infesta- | infesta- |
| Test solution | tion | tion | tion |
| Control (Acetonitrile 5%) | 1.7% | 1.7% | 1.7% |
| SEQ ID NO: 5 (GS-ω/κ- | 1.7% | 1.7% | 1.7% |
| Hxtx-Hv1h) (5000 ppm) | | | |
| SEQ ID NO: 5 (GS-ω/κ- | 0% | 0% | 0% |
| Hxtx-Hv1h) (2500 ppm) | | | |
| SEQ ID NO: 5 (GS-ω/κ- | 0% | 0% | 2% |
| Hxtx-Hv1h) (1250 ppm) | | | |
| SEQ ID NO: 5 (GS-ω/κ- | 0% | 0% | 0% |
| Hxtx-Hv1h) (625 ppm) | | | |
| SEQ ID NO: 1 (kB1) | 5% | 25% | 36% |
| (10000 ppm) | | | |
| SEQ ID NO: 5 (GS-ω/κ- | 5% | 46% | 59% |
| Hxtx-Hv1h) (5000 ppm) + | | | |
| SEQ ID NO: 1 (kB1) | | | |
| (10000 ppm) | | | |
| SEQ ID NO: 5 (GS-ω/κ- | 5% | 59% | 66% |
| Hxtx-Hv1h) (2500 ppm) + | | | |
| SEQ ID NO: 1 (kB1) | | | |
| (10000 ppm) | | | |
| SEQ ID NO: 5 (GS-ω/κ- | 0% | 46% | 60% |
| Hxtx-Hv1h) (1250 ppm) + | | | |
| SEQ ID NO: 1 (kB1) | | | |
| (10000 ppm) | | | |
| SEQ ID NO: 5 (GS-ω/κ- | 0% | 49% | 66% |
| Hxtx-Hv1h) (625 ppm) + | | | |
| SEQ ID NO: 1 (kB1) | | | |
| (10000 ppm) | | | |

### REFERENCES

Cranfield C. G., Henriques S. T., Martinac B., Duckworth P., Craik D. J., Cornell B.; Kalata B1 and Kalata B2 Have a Surfactant-Like Activity in Phosphatidylethanolomine-Containing Lipid Membranes; Langmuir, 33, 26, 6630-6637, 2017.
https://doi.org/10.1021/acs.langmuir.7b01642
Czalpa T. H. and Lang B. A.; Effect of Plant Lectins on the Larval Development of European Corn Borer (Lepidoptera: Pyralidae) and Southern Corn Rootworm (Coleoptera: Chrysomelidae); Journal of Economic Entomology, 83, 6, 2480-2485, 1990.
https://doi.org/10.1093/jee/83.6.2480
Gfeller D., Michielin O., Zoete V.; SwissSidechain: a molecular and structural database of non-natural sidechains; Nucleic Acids Research, 41, D1, D327-D332, 2013.
https://doi.org/10.1093/nar/gks991
Huang Y., Colgrave M. L., Clark R. J., Kotze A. C., Craik D. J.; Lysine-scanning Mutagenesis Reveals an Amendable Face of the Cyclotide Kalata B1 for the Optimization of Nematocidal Activity; J Biol. Chem, 285, 14, 10797-10805, 2010.
https://doi: 10.1074/jbc.M109.089854
Marrone P. G., Ferri F. D., Mosley T. R., Meinke L. J.; Improvements in Laboratory Rearing of the Southern Corn Rootworm, Diabrotica undecimpuncta howardi Barber (Coleoptera: Chrysomelidae), on an Artificial Diet and Corn; Journal of Economic Entomology, 78, 1, 290-293, 1985. https://doi.org/10.1093/jee/78.1.290
Savary S., Willocquet L., Pethybridge S. J., Esker P., McRoberts N., Nelson A.; The global burden of pathogens and pests on major food crops; Nat Ecol Evol. 2019 March; 3(3):430-439, 2019. https://doi.org/10.1038/s41559-018-0793-y
Weidmann J. and Craik D. J.; Discovery, structure, function, and applications of cyclotides: circular proteins from plants; J Exp Bot. 2016 August; 67(16):4801-12, 2016. https://doi.org/10.1093/jxb/erw210

1. An insecticidal composition comprising:
a. at least one cyclic peptide comprising an amino acid sequence of any of SEQ ID NOs: 1, 2 or 6, or an amino acid sequence that is at least 90%, 91%, 92%, 93%, 94%, 95%, 96%, 97%, 98% or 99% identical to any of SEQ ID NOs: 1, 2 or 6; and
b. at least one second peptide comprising an amino acid sequence of any of SEQ ID NOs: 3 to 5, or an amino acid sequence that is at least 90%, 91%, 92%, 93%, 94%, 95%, 96%, 97%, 98% or 99% identical to any of SEQ ID NOs: 3 to 5, or a variant of a peptide comprising an amino acid sequence of SEQ ID NO: 3, wherein the amino acid sequence has an amino acid variant on position N29, and which retains the biological activity of the peptide.

2. The composition according to claim 1, wherein the cyclic peptide comprises an amino acid sequence of any of: SEQ ID NOs: 1, 2 or 6; and the second peptide comprises an amino acid sequence of any of: SEQ ID NOs: 3, 4 or 5, or a variant of a peptide comprising an amino acid sequence of SEQ ID NO: 3, wherein the amino acid sequence has an amino acid variant on position N29, and which retains the biological activity of the peptide.

3. The composition according to claim 1, wherein the amino acid variant on position N29 is A, L, P or T.

4. The composition according to claim 1, wherein the cyclic peptide comprises an amino acid sequence of any of: SEQ ID NOs: 1, 2 or 6; and the second peptide comprises an amino acid sequence of any of: SEQ ID NOs: 3, 4 or 5.

5. The composition according to claim 1, wherein the cyclic peptide and/or the second peptide each have individually insecticidal activity when ingested by an insect.

6. A composition comprising an insecticidally effective amount of a peptide according to claim 1 and a suitable carrier or diluent therefor.

7. A plant, plant tissue, or plant propagation material comprising the composition according to claim 1, or having the composition according to claim 1 applied thereto.

8. A method for delivering a composition to an insect, the method comprising applying the composition according to claim 1 to the plant, plant locus, plant tissue or plant propagation material, to obtain a treated material suitable for contacting of, or ingestion by insects.

9. A method for controlling insect infestation and/or damage by insect pests, comprising applying a composition according to claim 1 to an insect or its environment, a plant, plant propagation material or the locus where the plant or plant propagation material is planted.

10. The method according to claim 9, wherein the composition comprises a cyclic peptide and a second peptide both individually having insecticidal activity when ingested by an insect.

11. Use of a composition according to claim 1 for controlling insect infestation.


SEQUENCE LISTING (7 sequences)

SEQ ID NO: 1 - protein, 29 aa, Oldenlandia affinis
GLPVCGETCVGGTCNTPGCTCSWPVCTRN

SEQ ID NO: 2 - protein, 29 aa, Oldenlandia affinis
GLPVCGETCFGGTCNTPGCSCTWPICTRD

SEQ ID NO: 3 - protein, 37 aa, Hadronyche versuta
SPTCIPSGQPCPYNENCCSQSCTFKENENGNTVKRCD

SEQ ID NO: 4 - protein, 27 aa, Anemonia sulcata
RSCCPCYWGGCPWGQNCYPEGCSGPKV

SEQ ID NO: 5 - protein, 41 aa, Artificial Sequence
  GS-w/k-hexatoxin-Hv1h
GSQYCVPVDQPCSLNTQPCCDDATCTQERNENGHTVYYCRA

SEQ ID NO: 6 - protein, 29 aa, Artificial Sequence
  Mutated kB1 (kB1[V25K])
GLPVCGETCVGGTCNTPGCTCSWPKCTRN

SEQ ID NO: 7 - protein, 37 aa, Atrax robustus
SPTCIPSGQPCPYNENCCSQSCTFKENETGNTVKRCD